(19) United States
(12) Patent Application Publication
(10) Pub. No.: US 2013/0280175 A1
(43) Pub. Date: Oct. 24, 2013

(54) WATER-CONTAINING ANTIPERSPIRANT COMPOSITIONS WITH IMPROVED WHITE MARK PROTECTION

(71) Applicant: Kenkel GA & Co. KGaA, Duesseldorf (DE)

(72) Inventors: Bernhard Banowski, Duesseldorf (DE); Nadine Schneider, Leverkusen (DE); Imme Breuer, Duesseldorf (DE)

(21) Appl. No.: 13/918,287

(22) Filed: Jun. 14, 2013

Related U.S. Application Data
(63) Continuation of application No. PCT/EP2011/071727, filed on Dec. 5, 2011.

(30) Foreign Application Priority Data
Dec. 16, 2010 (DE) 10 2010 063 250.3

(51) Int. Cl. A61K 8/34; A61Q 15/00

(52) U.S. Cl. CPC A61K 8/345; A61Q 15/00
USPC 424/43; 424/65; 424/66; 424/67; 424/68

(57) ABSTRACT

Antiperspirant compositions include at least one antiperspirant active substance, at least 5 wt. % free water and 0.5-15 wt. % 4-dimethylolcyclohexane.

### FIELD OF THE INVENTION

The present invention generally relates to water-included antiperspirant compositions having improved white mark protection.

### BACKGROUND OF THE INVENTION

Commercial perspiration-inhibiting compositions, also referred to below as antiperspirants, contain as the active antiperspirant ingredient at least one water-soluble astringent inorganic and/or organic salt of aluminum, zinc or selected aluminum zirconium mixed salts. The active antiperspirant ingredients have no direct effect on the activity of the sweat glands but minimize sweat secretion by narrowing the discharge ducts. The Al salts inhibit perspiration on treated areas of skin by superficial blocking of the sweat gland ducts as a result of Al mucopolysaccharide deposits.

Antiperspirant compositions are generally applied and used in the axillary area. When the composition dries on the skin or on clothing which has come into contact with the skin after application of the antiperspirant, the antiperspirant salt often remains visible as a white residue. This occurs even with water-containing compositions in which the antiperspirant salt is initially present in dissolved form. The white residues are perceived by the user as a highly negative property of the product. For masking aluminum salt residues of water-containing compositions, both water-soluble components, such as 1,2-propylene glycol, and oils, in particular ester oils such as isopropyl palmitate or alkyl benzoate, are known in the prior art. These masking agents wet the antiperspirant salt and do not evaporate even after application onto the skin, as do water and cyclomethicone, for example. As a result, the antiperspirant salt dries significantly more slowly and the occurrence of visible residues is delayed. The masking can be improved further by selecting a masking agent having a refractive index n_{D} in the range of the refractive index n_{D} of typical antiperspirant salts or typically used aqueous solutions of antiperspirant salts, i.e. in the range of n_{D}²⁰=1.4 to 1.5. The disadvantage of the known water-soluble masking agents, such as e.g. sorbitol and highly concentrated aqueous sorbitol solutions (50 to 70 wt. % sorbitol), lies in particular in the fact that they impart a sticky skin feel to the antiperspirant composition. Oils with a high masking action have the disadvantage that they can lead to fabric staining. The use of masking oils in formulations with a high proportion of aqueous phase is limited because of the difficulty of incorporating them, compatibility with the formulation and stability of the formulation. For formulations with a high proportion of aqueous phase and a low proportion of oil phase, in particular roll-on formulations, masking takes place substantially by masking agents for the aqueous phase, leading to particularly high stickiness of the formulation with the requisite high concentrations.

In formulations with a high proportion of aqueous phase, there is often also the problem of a lack of temperature stability, in particular at very low temperatures.

Moreover, in formulations with a high proportion of aqueous phase there is often the problem of poor perfume adhesion. Good perfume adhesion is particularly important for the commercial success of an antiperspirant product, since the consumer equates it with the antiperspirant action of the product. To improve the perfume adhesion of water-containing compositions, cationic surfactants are often used, but these can have a negative effect on the product's skin compatibility. The use of encapsulated perfume also prolongs perfume adhesion but increases the raw materials costs.

Water-containing antiperspirant compositions are available in various presentations, for example as a composition which can be sprayed with propellant, in particular as a water-in-oil emulsion which can be sprayed with propellant. These compositions are usually housed in spray cans made from aluminum or (less frequently) tinplate, which are protected from corrosion by internal lacquering. Despite this protective lacquering, however, corrosion damage can always occur. Another problem with these products lies in the fact that the valve becomes blocked. A composition which inhibits corrosion and/or reduces valve blockage would therefore be desirable for these specific presentations.

A constant need therefore exists for water-containing antiperspirant compositions with high residue masking, reduced visible residues, reduced stickiness, reduced fabric staining, improved temperature stability, prolonged perfume adhesion, and—in the case of sprayable compositions—a reduced corrosion effect and reduced valve blockage.

U.S. Pat. No. 7,569,530 discloses 1,2-cyclohexanedimethanol as an active antimicrobial substance. In U.S. Pat. No. 7,591,861, cyclohexanedimethanol (without any indication of the substitution positions) is disclosed as a glycolic solvent with a log P value of 0.5 to 4.0 for hair pretreatment, by means of which the penetration of a subsequently applied dye into the hair is improved.

It is therefore desirable to provide water-containing antiperspirant compositions with high residue masking and reduced visible residues.

It is therefore further desirable to provide water-containing antiperspirant compositions with reduced stickiness.

It is further desirable to provide water-containing antiperspirant compositions with reduced fabric staining.

It is further desirable to provide water-containing antiperspirant compositions with improved temperature stability, in particular at low temperatures.

It is further desirable to provide water-containing antiperspirant compositions with prolonged perfume adhesion.

It is further desirable to provide water-containing antiperspirant compositions with a reduced corrosion effect.

It is further desirable to provide water-containing antiperspirant compositions with a reduced valve blocking effect.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

An antiperspirant composition, including at least one active antiperspirant ingredient, at least 5 wt. % free water and 0.5-15 wt. % 1,4-dimethylolcyclohexane, wherein all quantitative data relate to the total weight of the antiperspirant composition, without taking into account any added propellant.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Surprisingly, it has been found that the above objects are achieved by 1,4-dimethylolcyclohexane (1,4-cyclohexanedimethanol).

The present application therefore provides an antiperspirant composition containing at least one active antiperspirant ingredient, at least 5 wt. % free water and 0.5-15 wt. % 1,4-dimethylolcyclohexane, all quantitative data being based on the total weight of the antiperspirant composition.

The present application also provides the use of 1,4-dimethylolcyclohexane in an antiperspirant composition containing at least one active antiperspirant ingredient and at least 5 wt. % free water to reduce the visibility of residues, all quantitative data being based on the total weight of the antiperspirant composition.

The present application also provides the use of 1,4-dimethylolcyclohexane in an antiperspirant composition containing at least one active antiperspirant ingredient and at least 5 wt. % free water to reduce the tack of the composition, all quantitative data being based on the total weight of the antiperspirant composition.

The present application also provides the use of 1,4-dimethylolcyclohexane in an antiperspirant composition containing at least one active antiperspirant ingredient and at least 5 wt. % free water to reduce fabric staining, all quantitative data being based on the total weight of the antiperspirant composition.

The present application also provides the use of 1,4-dimethylolcyclohexane in an antiperspirant composition containing at least one active antiperspirant ingredient and at least 5 wt. % free water to improve temperature stability, in particular at low temperatures, all quantitative data being based on the total weight of the antiperspirant composition.

The present application also provides the use of 1,4-dimethylolcyclohexane in an antiperspirant composition containing at least one active antiperspirant ingredient and at least 5 wt. % free water to prolong perfume adhesion, all quantitative data being based on the total weight of the antiperspirant composition.

The present application also provides the use of 1,4-dimethylolcyclohexane in an antiperspirant composition containing at least one active antiperspirant ingredient and at least 5 wt. % free water to reduce the corrosion effect, all quantitative data being based on the total weight of the antiperspirant composition.

The present application also provides the use of 1,4-dimethylolcyclohexane in an antiperspirant composition containing at least one active antiperspirant ingredient and at least 5 wt. % free water, which is contained in an aerosol dispensing device, to reduce the valve-blocking effect, all quantitative data being based on the total weight of the antiperspirant composition.

“Normal conditions” within the meaning of the present application are a temperature of 20° C. and a pressure of 1013.25 mbar. Melting point data also refer to a pressure of 1013.25 mbar.

All quantitative data, unless otherwise specified, are based on the total weight of the antiperspirant composition according to the invention. Any added propellants are not included in the antiperspirant composition according to the invention, and therefore all quantitative data are based on the total weight of the propellant-free antiperspirant composition, unless otherwise specified.

### Free Water

“Free water” within the meaning of the present application is water which is not contained in the antiperspirant composition in the form of water of crystallization, water of hydration or similarly molecularly bound water. The content of water of crystallization, water of hydration or similarly molecularly bound water which is contained in the components used, in particular in the active antiperspirant ingredients, does not represent free water within the meaning of the present application. Free water is e.g. water which is added to the composition according to the invention as a solvent, as a gel activator or as a solvent component of other active substances.

The antiperspirant compositions according to the invention contain, based on their total weight, at least 5 wt. % free water. Preferred antiperspirant compositions according to the invention contain, based on their total weight, 10 to 95 wt. % free water, preferably 20-80 wt. %, particularly preferably 30-70 wt. %, extraordinarily preferably 40-60 wt. % free water.

1,4-Dimethylolcyclohexane has the following structural formula:

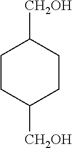

For the effects according to the invention, it is unimportant whether cyclohexane-1,4-dimethanol is used as a pure cis isomer, as a pure trans isomer or as an isomer mixture of cis and trans isomers.

Commercially available 1,4-dimethylolcyclohexane is present as a cis/trans isomer mixture. This isomer mixture has the CAS No. 105-08-8. A preferred isomer mixture according to the invention contains 29-33 wt. % cis isomer (CAS No. 3236-47-3) and 71-67 wt. % trans isomer (CAS No. 3236-48-4) and has a refractive index n_{D}²⁰ of 1.487.

Preferred antiperspirant compositions according to the invention contain, based on their total weight, 1 to 12 wt. %, preferably 2-10 wt. %, particularly preferably 3-7 wt. % 1,4-dimethylolcyclohexane.

### Active Antiperspirant Ingredients

Preferred active antiperspirant ingredients are selected from the water-soluble astringent inorganic and organic salts of aluminum, zirconium and zinc or any mixtures of these salts.

According to the invention, water solubility is understood to be a solubility of at least 3 wt. % at 20° C., i.e. quantities of at least 3 g of the active antiperspirant ingredient are soluble in 97 g water at 20° C.

According to the invention, water solubility is preferably understood to be a solubility of at least 5 wt. % at 20° C., i.e. quantities of at least 5 g of the active antiperspirant ingredient are soluble in 95 g water at 20° C.

Particularly preferred active antiperspirant ingredients are selected from aluminum chlorohydrate, in particular aluminum chlorohydrate with the general formula [Al₂(OH)₅CI.1-6H₂O]ₙ, preferably [Al₂(OH)₅CI.2-3H₂O]ₙ, which can be present in non-activated or in activated (depolymerized) form, and aluminum chlorohydrate with the general formula [Al₂(OH)₄Cl₂.1-6H₂O]ₙ, preferably [Al₂(OH)₄Cl₂.2-3H₂O]ₙ, which can be present in non-activated or in activated (depolymerized) form.

The production of preferred active antiperspirant ingredients is disclosed e.g. in U.S. Pat. No. 3,887,692, U.S. Pat. No. 3,904,741, U.S. Pat. No. 4,359,456, GB 2048229 and GB 1347950.

Also preferred are aluminum sesquichlorohydrate, aluminum dichlorohydrate, aluminum chlorohydrex propylene glycol (PG) or aluminum chlorohydrex polyethylene glycol (PEG), aluminum or aluminum zirconium glycol complexes, e.g. aluminum or aluminum zirconium propylene glycol complexes, aluminum sesquichlorohydrex PG or aluminum sesquichlorohydrex PEG, aluminum PG dichlorohydrex or aluminum PEG dichlorohydrex, aluminum hydroxide, also selected from the aluminum zirconium chlorohydrates, such as aluminum zirconium trichlorohydrate, aluminum zirconium tetrachlorohydrate, aluminum zirconium pentachlorohydrate, aluminum zirconium octachlorohydrate, the aluminum zirconium chlorohydrate glycine complexes, such as aluminum zirconium trichlorohydrex glycine, aluminum zirconium tetrachlorohydrex glycine, aluminum zirconium pentachlorohydrex glycine, aluminum zirconium octachlorohydrex glycine, potassium aluminum sulfate (KAl(SO₄)₂.12H₂O, alum), dehydrated alum (KAl(SO₄)₂ with zero to 11 moles of water of crystallization), aluminum undecylenoyl collagen amino acid, sodium aluminum lactate+aluminum sulfate, sodium aluminum chlorohydroxylactate, aluminum bromohydrate, aluminum chloride, the aluminum salts of lipoamino acids, aluminum sulfate, aluminum lactate, aluminum chlorohydroxyallantoinate, sodium aluminum chlorohydroxylactate, zinc chloride, zinc sulfocarbolate, zinc sulfate, zirconyl oxyhalides, in particular zirconyl oxychlorides, zirconyl hydroxyhalides, in particular zirconyl hydroxychlorides (zirconium chlorohydrate).

Particularly preferred active antiperspirant ingredients according to the invention are selected from so-called “activated” aluminum and aluminum zirconium salts, which are also referred to as active antiperspirant ingredients “with enhanced activity”. These active ingredients are known in the prior art and are commercially available. Their production is disclosed e.g. in GB 2048229, U.S. Pat. No. 4,775,528 and U.S. Pat. No. 6,010,688. Activated aluminum and aluminum zirconium salts are generally produced by heat treatment of a relatively dilute solution of the salt (e.g. approx. 10 wt. % salt) in order to increase its HPLC peak 4 to peak 3 area ratio. The activated salt can then be dried to a powder, in particular spray-dried. In addition to spray-drying, e.g. roller drying is also suitable.

Activated aluminum and aluminum zirconium salts typically have an HPLC peak 4 to peak 3 area ratio of at least 0.4, preferably at least 0.7, particularly preferably at least 0.9, wherein at least 70% of the aluminum can be assigned to these peaks.

Activated aluminum and aluminum zirconium salts do not necessarily have to be used as a spray-dried powder. Other preferred active antiperspirant ingredients according to the invention are non-aqueous solutions or solubilizates of an activated antiperspirant aluminum or aluminum zirconium salt, e.g. according to U.S. Pat. No. 6,010,688, which are stabilized by the addition of an effective quantity of a polyhydric alcohol having 3 to 6 carbon atoms and 3 to 6 hydroxyl groups, preferably propylene glycol, sorbitol and pentaerythritol, against loss of activation against the rapid degradation of the HPLC peak 4:peak 3 area ratio of the salt. For example, compositions are preferred which contain in weight percent (USP): 18-45 wt. % of an activated aluminum or aluminum zirconium salt, 55-82 wt. % of at least one water-free polyhydric alcohol having 3 to 6 carbon atoms and 3 to 6 hydroxyl groups, preferably propylene glycol, butylene glycol, diethylene glycol, dipropylene glycol, glycerol, sorbitol and pentaerythritol, particularly preferably propylene glycol.

Also particularly preferred are complexes of activated antiperspirant aluminum or aluminum zirconium salts with a polyhydric alcohol, which contain 20-50 wt. %, particularly preferably 20-42 wt. %, activated antiperspirant aluminum or aluminum zirconium salt and 2-16 wt. % molecularly bound water, wherein the balance to 100 wt. % is at least one polyhydric alcohol having 3 to 6 carbon atoms and 3 to 6 hydroxyl groups. Propylene glycol, propylene glycol/sorbitol mixtures and propylene glycol/pentaerythritol mixtures are preferred alcohols of this type. Such preferred complexes of an activated antiperspirant aluminum or aluminum zirconium salt with a polyhydric alcohol according to the invention are disclosed e.g. in U.S. Pat. No. 5,643,558 and U.S. Pat. No. 6,245,325.

Other preferred active antiperspirant ingredients are calcium aluminum basic salts, as disclosed e.g. in U.S. Pat. No. 2,571,030. These salts are produced by reacting calcium carbonate with aluminum chlorhydroxide or aluminum chloride and aluminum powder or by adding calcium chloride dihydrate to aluminum chlorhydroxide.

Other preferred active antiperspirant ingredients are aluminum zirconium complexes, as disclosed e.g. in U.S. Pat. No. 4,017,599, which are buffered with salts of amino acids, in particular with alkali and alkaline earth metal glycinates.

Other preferred active antiperspirant ingredients are activated aluminum or aluminum zirconium salts, as disclosed e.g. in U.S. Pat. No. 6,245,325 or U.S. Pat. No. 6,042,816, containing 5-78 wt. % (USP) of an activated antiperspirant aluminum or aluminum zirconium salt, an amino acid or hydroxyalkanoic acid in a sufficient quantity to provide an (amino acid or hydroxyalkanoic acid) to (Al+Zr) weight ratio of 2:1-1:20 and preferably 1:1 to 1:10, and a water-soluble calcium salt in a sufficient quantity to provide a Ca:(Al+Zr) weight ratio of 1:1-1:28 and preferably 1:2-1:25. Particularly preferred solid activated antiperspirant salt compositions, e.g. according to U.S. Pat. No. 6,245,325 or U.S. Pat. No. 6,042,816, contain 48-78 wt. % (USP), preferably 66-75 wt. %, of an activated aluminum or aluminum zirconium salt and 1-16 wt. %, preferably 4-13 wt. %, molecularly bound water (water of hydration), and also sufficient water-soluble calcium salt that the Ca:(Al+Zr) weight ratio is 1:1-1:28, preferably 1:2-1:25, and sufficient amino acid that the amino acid to (Al+Zr) weight ratio is 2:1-1:20, preferably 1:1-1:10.

Other particularly preferred solid antiperspirant activated salt compositions, e.g. according to U.S. Pat. No. 6,245,325 or U.S. Pat. No. 6,042,816, contain 48-78 wt. % (USP), preferably 66-75 wt. %, of an activated aluminum or aluminum zirconium salt and 1-16 wt. %, preferably 4-13 wt. %, molecularly bound water (water of hydration), and also sufficient water-soluble calcium salt that the Ca:(Al+Zr) weight ratio is 1:1-1:28, preferably 1:2-1:25, and sufficient glycine that the glycine to (Al+Zr) weight ratio is 2:1-1:20, preferably 1:1-1:10.

Other particularly preferred solid antiperspirant activated salt compositions, e.g. according to U.S. Pat. No. 6,245,325 or U.S. Pat. No. 6,042,816, contain 48-78 wt. % (USP), preferably 66-75 wt. %, of an activated aluminum or aluminum zirconium salt and 1-16 wt. %, preferably 4-13 wt. %, molecularly bound water, and also sufficient water-soluble calcium salt that the Ca:(Al+Zr) weight ratio is 1:1-1:28, preferably 1:2-1:25, and sufficient hydroxyalkanoic acid that the hydroxyalkanoic acid to (Al+Zr) weight ratio is 2:1-1:20, preferably 1:1-1:10.

Preferred water-soluble calcium salts for stabilizing the antiperspirant salts are selected from calcium chloride, calcium bromide, calcium nitrate, calcium citrate, calcium formate, calcium acetate, calcium gluconate, calcium ascorbate, calcium lactate, calcium glycinate, calcium carbonate, calcium sulfate, calcium hydroxide and mixtures thereof.

Preferred amino acids for stabilizing the antiperspirant salts are selected from glycine, alanine, leucine, isoleucine, β-alanine, valine, cysteine, serine, tryptophan, phenylalanine, methionine, β-amino-n-butanoic acid and γ-amino-n-butanoic acid and the salts thereof, in each case in the d-form, the I-form and the dl-form; glycine is particularly preferred.

Preferred hydroxyalkanoic acids for stabilizing the antiperspirant salts are selected from glycolic acid and lactic acid.

Other preferred active antiperspirant ingredients are activated aluminum or aluminum zirconium salts, as disclosed e.g. in U.S. Pat. No. 6,902,723, containing 5-78 wt. % (USP) of an activated antiperspirant aluminum or aluminum zirconium salt, an amino acid or hydroxyalkanoic acid in a sufficient quantity to provide an (amino acid or hydroxyalkanoic acid) to (Al+Zr) weight ratio of 2:1-1:20 and preferably 1:1 to 1:10, and a water-soluble strontium salt in a sufficient quantity to provide an Sr:(Al+Zr) weight ratio of 1:1-1:28 and preferably 1:2-1:25.

Particularly preferred solid antiperspirant activated salt compositions, e.g. according to U.S. Pat. No. 6,902,723, contain 48-78 wt. % (USP), preferably 66-75 wt. %, of an activated aluminum or aluminum zirconium salt and 1-16 wt. %, preferably 4-13 wt. %, molecularly bound water, and sufficient water-soluble strontium salt that the Sr:(Al+Zr) weight ratio is 1:1-1:28, preferably 1:2-1:25, and sufficient amino acid that the amino acid to (Al+Zr) weight ratio is 2:1-1:20, preferably 1:1-1:10.

Other particularly preferred solid antiperspirant activated salt compositions, e.g. according to U.S. Pat. No. 6,902,723, contain 48-78 wt. % (USP), preferably 66-75 wt. %, of an activated aluminum or aluminum zirconium salt and 1-16 wt. %, preferably 4-13 wt. %, molecularly bound water, and also sufficient water-soluble strontium salt that the Sr:(Al+Zr) weight ratio is 1:1-1:28, preferably 1:2-1:25, and sufficient glycine that the glycine to (Al+Zr) weight ratio is 2:1-1:20, preferably 1:1-1:10.

Other particularly preferred solid antiperspirant activated salt compositions, e.g. according to U.S. Pat. No. 6,902,723, contain 48-78 wt. % (USP), preferably 66-75 wt. %, of an activated aluminum or aluminum zirconium salt and 1-16 wt. %, preferably 4-13 wt. %, molecularly bound water, and also sufficient water-soluble strontium salt that the Sr:(Al+Zr) weight ratio is 1:1-1:28, preferably 1:2-1:25, and sufficient hydroxyalkanoic acid that the hydroxyalkanoic acid to (Al+Zr) weight ratio is 2:1-1:20, preferably 1:1-1:10.

Other preferred activated aluminum salts are those of the general formula Al₂(OH)₆₋ₐXa, where X is Cl, Br, I or NO₃ and “a” is a value of 0.3 to 5, preferably of 0.8 to 2.5 and particularly preferably 1 to 2, so that the molar ratio of Al:X is 0.9:1 to 2.1:1, as disclosed e.g. in U.S. Pat. No. 6,074,632. In these salts, some water of hydration is generally associatively bound, typically 1 to 6 moles of water per mole of salt. Particularly preferred is aluminum chlorohydrate (i.e. X is Cl in the aforementioned formula) and especially 5/6-basic aluminum chlorohydrate, where “a” is 1, so that the molar ratio of aluminum to chlorine is 1.9:1 to 2.1:1. Particularly preferred zirconium-free aluminum sesquichlorohydrates according to the invention have a molar metal to chloride ratio of 1.5:1-1.8:1.

Preferred activated aluminum zirconium salts are those which represent mixtures or complexes of the aluminum salts described above with zirconium salts of the formula ZrO(OH)_{2-pb}Y_{b}, where Y is Cl, Br, I, NO₃ or SO₄, b is a rational number from 0.8 to 2 and p is the valency of Y, as disclosed e.g. in U.S. Pat. No. 6,074,632. The zirconium salts generally also have some associatively bound water of hydration, typically 1 to 7 moles of water per mole of salt. The zirconium salt is preferably zirconyl hydroxychloride with the formula ZrO(OH)_{2-b}Cl_{b}, where b is a rational number from 0.8 to 2, preferably 1.0 to 1.9. Preferred aluminum zirconium salts have an Al:Zr molar ratio of 2 to 10 and a metal:(X+Y) ratio of 0.73 to 2.1, preferably 0.9 to 1.5. A particularly preferred salt is aluminum zirconium chlorohydrate (i.e. X and Y are Cl), which has an Al:Zr ratio of 2 to 10 and a molar metal:Cl ratio of 0.9 to 2.1. The term aluminum zirconium chlorohydrate encompasses the tri-, tetra-, penta- and octachlorohydrate forms.

Preferred zirconium salts according to the invention have the general formula ZrO(OH)₂₋ₐClₐ.x H₂O with a=1.5-1.87; x=1-7, wherein a and x are rational numbers. These zirconium salts are disclosed e.g. in the Belgian document BE 825146.

Other preferred active antiperspirant ingredients are disclosed in U.S. Pat. No. 6,663,854 and US 20040009133.

The active antiperspirant ingredients can be present both in dissolved form and in solubilized form. The active antiperspirant ingredients can be used as non-aqueous solutions or as glycolic solubilizates.

Preferred aluminum zirconium salts have a molar metal to chloride ratio of 0.9-1.3, preferably 0.9-1.1, particularly preferably 0.9-1.0.

Preferred aluminum zirconium chlorohydrates generally have the empirical formula AlₙZr(OH)_{[3n+4-m(n+1)]}(Cl)_{[m(n+1)]} with n=2.0-10.0, preferably 3.0-8.0, m=0.77-1.11 (corresponding to a molar metal (Al+Zr) to chloride ratio of 1.3-0.9), preferably m=0.91-1.11 (corresponding to M:CI=1.1-0.9), and particularly preferably m=1.00-1.11 (corresponding to M:CI=1.0-0.9), and also very preferably m=1.02-1.11 (corresponding to M:CI=0.98-0.9) and very preferably m=1.04-1.11 (corresponding to M:CI=0.96-0.9).

In these salts there is generally some associatively bound water of hydration, typically 1-6 moles of water per mole of salt, corresponding to 1-16 wt. %, preferably 4-13 wt. %, water of hydration.

The preferred aluminum zirconium chlorohydrates are usually associated with an amino acid in order to prevent the polymerization of the zirconium species during production. Preferred stabilizing amino acids are selected from glycine, alanine, leucine, isoleucine, β-alanine, cysteine, valine, serine, tryptophan, phenylalanine, methionine, β-amino-n-butanoic acid and γ-amino-n-butanoic acid and the salts thereof, in each case in the d-form, the I-form and the dl-form; glycine is particularly preferred. The amino acid is contained in the salt in a quantity of 1-3 mol, preferably 1.3-1.8 mol, per mole of zirconium in each case.

Preferred antiperspirant salts are aluminum zirconium tetrachlorohydrate (Al:Zr=2-6; M:Cl=0.9-1.3), in particular salts with a molar metal to chloride ratio of 0.9-1.1, preferably 0.9-1.0.

Also preferred according to the invention are aluminum zirconium chlorohydrate glycine salts which are stabilized with betaine ((CH₃)₃N⁺—CH₂—COO⁻). Particularly preferred corresponding compounds have an overall molar (betaine+glycine)/Zr ratio of (0.1-3.0):1, preferably (0.7-1.5):1, and a molar ratio of betaine to glycine of at least 0.001:1. Corresponding compounds are disclosed e.g. in U.S. Pat. No. 7,105,691.

In a particularly preferred embodiment according to the invention, a so-called “activated” salt is contained as a particularly effective antiperspirant salt, in particular one with a high HPLC peak 5 aluminum content, in particular with a peak 5 area of at least 33%, particularly preferably at least 45%, based on the total area under peaks 2-5, measured using HPLC in a 10 wt. % aqueous solution of the active substance under conditions in which the aluminum species are resolved into at least 4 consecutive peaks (referred to as peaks 2-5). Preferred aluminum zirconium salts with a high HPLC peak 5 aluminum content (also referred to as “E⁵AZCH”) are disclosed e.g. in U.S. Pat. No. 6,436,381 and U.S. Pat. No. 6,649,152.

Those activated “E⁵AZCH” salts of which the HPLC peak 4 to peak 3 area ratio is from at least 0.4, preferably at least 0.7, particularly preferably at least 0.9, are also preferred.

Other particularly preferred active antiperspirant ingredients are those aluminum zirconium salts with a high HPLC peak 5 aluminum content which are additionally stabilized with a water-soluble strontium salt and/or with a water-soluble calcium salt. Corresponding salts are disclosed e.g. in U.S. Pat. No. 6,923,952.

Particularly preferred compositions according to the invention are characterized in that the at least one active antiperspirant ingredient is contained in a quantity of 5-40 wt. %, preferably 10-35 wt. %, particularly preferably 15-28 wt. % and extraordinarily preferably 23-27 wt. %, based on the total weight of the active substance (USP) free from water of crystallization in the overall composition.

Since the residue-masking action of 1,4-dimethylolcyclohexane can be used particularly advantageously with aluminum zirconium salts, preferred compositions according to the invention contain at least one aluminum zirconium salt as active antiperspirant ingredient, at least 5 wt. % free water and 0.5-15 wt. % 1,4-dimethylolcyclohexane, all quantitative data being based on the total weight of the antiperspirant composition.

In another particularly preferred embodiment, the compositions according to the invention can contain both at least one active deodorant ingredient and at least one active antiperspirant ingredient.

In order to improve further the antiperspirant and deodorant action of the compositions according to the invention, in a preferred embodiment these contain at least one active deodorant ingredient.

Preferred active deodorant ingredients according to the invention are odor absorbers, ion exchangers having a deodorizing action, bacteriostatic agents, components having a prebiotic action and enzyme inhibitors or, particularly preferably, combinations of the above active ingredients.

Silicates act as odor absorbers, which at the same time also advantageously support the rheological properties of the composition according to the invention. The particularly preferred silicates according to the invention primarily include layered silicates and among these in particular montmorillonite, kaolinite, illite, beidellite, nontronite, saponite, hectorite, bentonite, smectite and talcum. Other preferred odor absorbers are e.g. zeolites, zinc ricinoleate, cyclodextrins, certain metal oxides, such as e.g. aluminum oxide, and chlorophyll. They are preferably used in a quantity of 0.1-10 wt. %, particularly preferably 0.5-7 wt. % and extraordinarily preferably 1-5 wt. %, based in each case on the overall composition.

Other preferred odor absorbers according to the invention are selected from perlite (volcanic glass) and expanded perlite.

Bacteriostatic or antimicrobial active ingredients are understood according to the invention to be those active ingredients which reduce the number of skin microbes involved in odor formation or inhibit their growth. These microbes include inter alia various species from the group of the staphylococci, the group of the corynebacteria, anaerococci and micrococci.

As bacteriostatic or antimicrobial active ingredients, in particular organohalogen compounds and organohalides, quaternary ammonium compounds, a series of plant extracts and zinc compounds are preferred according to the invention. These include inter alia triclosan, chlorhexidine and chlorhexidine gluconate, 3,4,4′-trichlorocarbanilide, bromochlorophen, dichlorophen, chlorothymol, chloroxylenol, hexachlorophene, dichloro-m-xylenol, dequalinium chloride, domiphen bromide, ammonium phenolsulfonate, benzalkonium halides, benzalkonium cetyl phosphate, benzalkonium saccharinates, benzethonium chloride, cetylpyridinium chloride, lauryl pyridinium chloride, lauryl isoquinolinium bromide and methylbenzethonium chloride. It is also possible to use phenol, phenoxyethanol, disodium dihydroxyethyl sulfosuccinyl undecylenate, sodium bicarbonate, zinc lactate, sodium phenolsulfonate and zinc phenolsulfonate, ketoglutaric acid, terpene alcohols, such as e.g. farnesol, chlorophyllin copper complexes, α-monoalkyl glycerol ethers with a branched or linear, saturated or unsaturated, optionally hydroxylated C₆-C₂₂ alkyl residue, particularly preferably α-(2-ethylhexyl)glycerol ether, commercially available as Sensiva® SC 50 (ex Schülke & Mayr), carboxylic acid esters of mono-, di- and triglycerol (e.g. glycerol monolaurate, diglycerol monocaprinate), lantibiotics and plant extracts (e.g. green tea and components of linden blossom oil).

Other preferred active deodorant ingredients are selected from so-called prebiotically active components, which are understood according to the invention to be those components which inhibit only or at least predominantly the odor-forming microbes of the skin microflora, but not the desirable, i.e. non-odor-forming, microbes which are part of a healthy skin microflora. These explicitly include here the active ingredients which are disclosed as prebiotically active in published patent applications DE 10333245 and DE 10 2004 011 968; they include conifer extracts, in particular from the group of the Pinaceae, and plant extracts from the group of the Sapindaceae, Araliaceae, Lamiaceae and Saxifragaceae, in particular extracts from Picea spp., Paullinia sp., Panax sp., Lamium album or Ribes nigrum and mixtures of these substances.

Other preferred active deodorant ingredients are selected from the perfume oils having a bacteriostatic action and the Deosafe® perfume oils, which are available from Symrise, formerly Haarmann & Reimer.

Other preferred active deodorant ingredients are selected from silver salts, in particular silver citrate, dihydrogen silver citrate, silver lactate and silver sulfate, soluble complex salts of silver, colloidal silver and silver zeolites.

The enzyme inhibitors include substances which inhibit the enzymes responsible for breaking down sweat, in particular arylsulfatase, β-glucuronidase, aminoacylase, esterases, lipases and/or lipoxygenase, e.g. trialkyl citric acid esters, in particular triethyl citrate, or zinc glycinate.

Preferred antiperspirant compositions according to the invention are characterized in that the at least one active deodorant ingredient is selected from arylsulfatase inhibitors, beta-glucuronidase inhibitors, aminoacylase inhibitors, esterase inhibitors, lipase inhibitors and lipoxygenase inhibitors, α-monoalkyl glycerol ethers with a branched or linear, saturated or unsaturated, optionally hydroxylated C₆-C₂₂ alkyl residue, in particular α-(2-ethylhexyl)glycerol ether, phenoxyethanol, perfume oils having a bacteriostatic action, Deosafe® perfume oils (Deosafe® is a registered trade mark of Symrise, formerly Haarmann & Reimer), prebiotically active components, trialkyl citric acid esters, in particular triethyl citrate, active ingredients which reduce the number of skin microbes involved in odor formation from the group of the staphylococci, corynebacteria, anaerococci and micrococci or inhibit the growth thereof, zinc compounds, in particular zinc phenolsulfonate and zinc ricinoleate, organohalogen compounds, in particular triclosan, chlorhexidine, chlorhexidine gluconate and benzalkonium halides, quaternary ammonium compounds, in particular cetylpyridinium chloride, odor absorbers, in particular silicates and zeolites, sodium bicarbonate, lantibiotics and mixtures of the aforementioned substances.

Other preferred antiperspirant compositions according to the invention are characterized in that the at least one active deodorant ingredient is contained in a total quantity of 0.1-10 wt. %, preferably 0.2-7 wt. %, particularly preferably 0.3-5 wt. % and extraordinarily preferably 0.4-1.0 wt. %, based in each case on the total weight of the active substance of the active deodorant ingredient or active deodorant ingredients in the overall composition.

In another particularly preferred embodiment, the compositions according to the invention contain both at least one active deodorant and at least one active antiperspirant ingredient.

Another preferred embodiment of the invention is characterized in that 0.01-5 wt. %, preferably 0.05-2 wt. %, particularly preferably 0.1-1 wt. %, based in each case on the total composition according to the invention, of a mixture of mineral substances obtained from a natural mineral water, a thermal water or a natural spa water is contained. Surprisingly, it has been found that these mixtures of mineral substances can further improve the antiperspirant performance of the compositions according to the invention. In addition, these mixtures of mineral substances can have a favorable effect on the skin compatibility of the compositions according to the invention. The name “natural mineral water” is based on the definition of the German Mineral and Table Waters Ordinance (MinTafWV, section 2). The solid residue of said waters is considered to be a mixture of mineral substances. Particularly preferred mixtures of mineral substances according to the invention come from the thermal water from La Toja (Spain), Bad Blumau, Bad Radkersburg, Aachen, Wiesbaden (all Germany), Carlsbad (Czech Republic), La Bourboule, Enghien-les-bains, Allevard-les-bains, Digne, Nyrac-les-bains, Lons le Saunier, Eaux Bonnes, Rochefort, les Fumades, Saint Christau, Uriage-les-bains, la Roche-Posay (all France) or the natural mineral waters or spa waters of Evian, Volvic, Vichy, Avène, Vittel (all France), Gerolstein or Fachingen (Germany).

Other preferred embodiments of the invention are characterized in that the composition according to the invention is present in stick form, as an aerosol spray, pump spray, liquid or gel roll-on application, cream, lotion, solution or gel.

Antiperspirant sticks can be present in gel form, on the basis of a W/O emulsion, on the basis of an O/W emulsion, on the basis of a water-oil multiple emulsion, on the basis of a nanoemulsion and on the basis of a microemulsion, wherein the oil phase can contain at least one silicone component or consist of at least one silicone component. Moreover, the compositions according to the invention which are formulated as antiperspirant sticks can be present on the basis of a polyol-in-oil emulsion, on the basis of an oil-in-polyol emulsion, on the basis of a polyol-oil multiple emulsion, on the basis of a nanoemulsion and on the basis of a microemulsion, wherein the polyol phase can have only a low water content (e.g. 5-10 wt. %, based on the total composition). Gel sticks can be formulated on the basis of fatty acid soaps, alditols, in particular dibenzylidene sorbitol, N-acylamino acid amides, 12-hydroxystearic acid, polyamides, polyamide derivatives, polysaccharides such as xanthan, polyglucomannans, guar, konjac, celluloses or starches, polyacrylates, polyacrylate derivatives and other gel formers.

Aerosol sprays, pump sprays, roll-on applications and creams can be present as a water-in-oil emulsion, water-in-silicone oil emulsions, an oil-in-water emulsion, silicone oil-in-water emulsion, water-in-oil microemulsion, oil-in-water microemulsion, silicone oil-in-water microemulsion, polyol-in-oil emulsion, oil-in-polyol emulsion, polyol-oil multiple emulsion, alcoholic solution, in particular ethanolic solution, hydroalcoholic solution, in particular solutions with more than 50 wt. % of a water-ethanol mixture, glycolic solution, in particular as a solution in propylene glycol, glycerol, dipropylene glycol and (under normal conditions) liquid polyethylene glycols, hydroglycolic solution, polyol solution, water-polyol solution and as an aqueous gel. All of the above compositions can be thickened, e.g. on the basis of fatty acid soaps, dibenzylidene sorbitol, N-acylamino acid amides, 12-hydroxystearic acid, polyacrylates of the carbomer and Carbopol type, polyacrylamides and polysaccharides, which can be chemically and/or physically modified.

The compositions according to the invention can be transparent, translucent or opaque. Where the compositions according to the invention are present in the form of a stick, they preferably contain a wax matrix encompassing at least one wax component with a melting point >50° C.

Preferred according to the invention are e.g. natural plant waxes, e.g. candelilla wax, carnauba wax, Japan wax, sugarcane wax, ouricury wax, cork wax, sunflower wax, fruit waxes, such as orange waxes, lemon waxes, grapefruit wax, and animal waxes, e.g. beeswax, shellac wax and cetaceum. Within the meaning of the invention, it can be particularly preferred to use hydrogenated or hardened waxes. It is also possible to use chemically modified waxes as a wax component, in particular the hard waxes, such as e.g. montan ester waxes, hydrogenated jojoba waxes and Sasol waxes. The synthetic waxes which are also preferred according to the invention include e.g. polyalkylene waxes and polyethylene glycol waxes, C₂₀-C₄₀ dialkyl esters of dimer acids, C₃₀₋₅₀ alkyl beeswax and alkyl and alkylaryl esters of dimer fatty acids.

A particularly preferred wax component is selected from at least one ester of a saturated, monohydric C₁₆-C₆₀ alcohol and a saturated C₈-C₃₆ monocarboxylic acid. According to the invention, these also include lactides, the cyclic double esters of alpha-hydroxycarboxylic acids with the appropriate chain length. Esters of fatty acids and long-chain alcohols have proved particularly advantageous for the composition according to the invention, because they impart excellent sensory properties to the antiperspirant preparation and high stability to sticks. The esters are composed of saturated, branched or unbranched monocarboxylic acids and saturated, branched or unbranched monohydric alcohols. Esters of aromatic carboxylic acids or hydroxycarboxylic acids (e.g. 12-hydroxystearic acid) and saturated, branched or unbranched alcohols can also be used according to the invention, provided that the wax component has a melting point >50° C. Particularly preferably, the wax components are selected from the group of the esters of saturated, branched or unbranched alkane carboxylic acids with a chain length of 12 to 24 C atoms and the saturated, branched or unbranched alcohols with a chain length of 16 to 50 C atoms having a melting point >50° C.

In particular, C₁₆₋₃₆ alkyl stearates and C₁₈₋₃₈ alkyl hydroxystearoyl stearates, C₂₀₋₄₀ alkyl erucates and cetearyl behenate can be advantageous as the wax component. The wax or the wax components have a melting point >50° C., preferably >60° C.

A particularly preferred embodiment according to the invention in stick form contains a C₂₀-C₄₀ alkyl stearate as a wax component. This ester is known by the names Kester Wax® K82H or Kester Wax® K80H and is marketed by Koster Keunen Inc. Another particularly preferred embodiment according to the invention in stick form contains cetearyl behenate as a wax component, i.e. mixtures of cetyl behenate and stearyl behenate. This ester is known by the name Kester Wax® K62 and is marketed by Koster Keunen Inc.

Other preferred wax components with a melting point >50° C. are the triglycerides of saturated and optionally hydroxylated C₁₂₋₃₀ fatty acids, such as hardened triglyceride fats (hydrogenated palm oil, hydrogenated coconut oil, hydrogenated castor oil), glyceryl tribehenate (tribehenin) or glyceryl tri-12-hydroxystearate, and also synthetic full esters of fatty acids and glycols or polyols having 2-6 carbon atoms, provided that they have a melting point higher than 50° C., e.g. preferably C₁₈-C₃₆ acid triglycerides (Syncrowax® HGL-C). Hydrogenated castor oil is particularly preferred as a wax component according to the invention.

Other preferred wax components with a melting point >50° C. are the saturated linear C₁₄-C₃₆ carboxylic acids, in particular myristic acid, palmitic acid, stearic acid and behenic acid and mixtures of these compounds, e.g. Syncrowax® AW 1C(C₁₈-C₃₆ fatty acids) or Cutina® FS 45 (palmitic and stearic acid).

Preferred antiperspirant compositions according to the invention, in particular antiperspirant sticks, contain, based in each case on the total weight of the propellant-free antiperspirant composition, at least one active antiperspirant ingredient, at least 5 wt. % free water, 0.5-15 wt. % 1,4-dimethylolcyclohexane and at least one wax component, which is selected from esters of a saturated monohydric C₁₆-C₆₀ alkanol and a saturated C₈-C₃₆ monocarboxylic acid, in particular cetyl behenate, stearyl behenate and C₂₀-C₄₀ alkyl stearate, glycerol triesters of saturated linear C₁₂-C₃₀ carboxylic acids, which may be hydroxylated, candelilla wax, carnauba wax, beeswax, saturated linear C₁₄-C₃₆ carboxylic acids and mixtures of the aforementioned substances, all quantitative data being based on the total weight of the antiperspirant composition. Particularly preferred antiperspirant compositions contain, based in each case on the total weight of the propellant-free antiperspirant composition, at least one active antiperspirant ingredient, at least 5 wt. % free water, 0.5-15 wt. % 1,4-dimethylolcyclohexane and a mixture of wax components, which is selected from mixtures of cetyl behenate, stearyl behenate, hydrogenated castor oil, palmitic acid and stearic acid. Other particularly preferred compositions contain at least one active antiperspirant ingredient, at least 5 wt. % free water, 0.5-15 wt. % 1,4-dimethylolcyclohexane and a mixture of wax components, which is selected from mixtures of C₂₀-C₄₀ alkyl stearate, hydrogenated castor oil, palmitic acid and stearic acid.

Preferred antiperspirant compositions according to the invention, in particular antiperspirant sticks, based on an oil-in-water emulsion contain at least one active antiperspirant ingredient, at least 5 wt. % free water, 0.5-15 wt. % 1,4-dimethylolcyclohexane and in total 4-20 wt. %, preferably 8-15 wt. %, of at least one wax component, which is selected from esters of a saturated monohydric C₁₆-C₆₀ alkanol and a saturated C₈-C₃₆ monocarboxylic acid, in particular cetyl behenate, stearyl behenate and C₂₀-C₄₀ alkyl stearate, glycerol triesters of saturated linear C₁₂-C₃₀ carboxylic acids, which may be hydroxylated, candelilla wax, carnauba wax, beeswax, saturated linear C₁₄-C₃₆ carboxylic acids and mixtures of the aforementioned substances, all quantitative data being based on the total weight of the antiperspirant composition. In a particularly preferred embodiment, the antiperspirant compositions according to the invention, in particular antiperspirant sticks, contain at least one active antiperspirant ingredient, at least 5 wt. % free water, 0.5-15 wt. % 1,4-dimethylolcyclohexane and at least one ester of a saturated monohydric C₁₆-C₆₀ alcohol and a saturated C₈-C₃₆ monocarboxylic acid, which represents a wax component, in a total quantity of 2-10 wt. %, preferably 2-6 wt. %, based in each case on the overall composition.

### Oil-in-Water Emulsifiers

The compositions according to the invention which are formulated as an emulsion, in particular as an oil-in-water emulsion or polyol-in-water emulsion, preferably contain at least one nonionic oil-in-water emulsifier with an HLB value of more than 7. These are emulsifiers which are generally known to the person skilled in the art, as listed e.g. in Kirk-Othmer, “Encyclopedia of Chemical Technology”, 3rd edition, 1979, volume 8, pages 913-916. For ethoxylated products, the HLB value is calculated by the formula HLB=(100-L):5, wherein L is the proportion by weight of lipophilic groups, i.e. fatty alkyl or fatty acyl groups, in the ethylene oxide adducts, expressed in weight percent.

In the selection of suitable nonionic oil-in-water emulsifiers according to the invention, it is particularly preferred to use a mixture of nonionic oil-in-water emulsifiers so that the stability of O/W emulsion compositions according to the invention can be optimally adjusted. The individual emulsifier components provide a proportion of the overall HLB value or average HLB value of the oil-in-water emulsifier mixture according to their proportion by weight in the total weight of the nonionic and optionally present ionic oil-in-water emulsifiers. According to the invention, the average HLB value of the oil-in-water emulsifier mixture is 10-19, preferably 12-18 and particularly preferably 14-17. In order to achieve these average HLB values, preferably oil-in-water emulsifiers from the HLB value ranges of 10-14, 14-16 and optionally 16-19 are combined with one another. In another preferred embodiment, the antiperspirant compositions according to the invention can also contain only a single nonionic oil-in-water emulsifier with an HLB value in the range of 10-19.

Preferred antiperspirant compositions according to the invention are characterized in that the nonionic oil-in-water emulsifiers are selected from ethoxylated C₈-C₂₄ alkanols with on average 10-100 moles ethylene oxide per mole, ethoxylated C₈-C₂₄-carboxylic acids with on average 10-100 moles ethylene oxide per mole, silicone copolyols with ethylene oxide units or with ethylene oxide and propylene oxide units, alkyl mono- and oligoglycosides with 8 to 22 carbon atoms in the alkyl residue and ethoxylated analogs thereof, ethoxylated sterols, partial esters of polyglycerols with 2 to 10 glycerol units and esterified with 1 to 4 saturated or unsaturated, linear or branched, optionally hydroxylated C₈-C₃₀ fatty acid residues, provided that they have an HLB value of more than 7, and mixtures of the aforementioned substances.

The ethoxylated C₈-C₂₄ alkanols have the formula R¹O(CH₂CH₂O)ₙH, wherein R¹ denotes a linear or branched alkyl and/or alkenyl residue with 8-24 carbon atoms and n the average number of ethylene oxide units per molecule, for numbers of 10-100, preferably 10-30 moles ethylene oxide to 1 mole capryl alcohol, 2-ethylhexyl alcohol, capric alcohol, lauryl alcohol, isotridecyl alcohol, myristyl alcohol, cetyl alcohol, isocetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol and technical-grade mixtures thereof. Adducts of 10-100 moles ethylene oxide to industrial fatty alcohols with 12-18 carbon atoms, such as e.g. coconut, palm, palm kernel or tallow fatty alcohol, are also suitable.

The ethoxylated C₈-C₂₄ carboxylic acids have the formula R¹(OCH₂CH₂)ₙOH, wherein R¹ denotes a linear or branched, saturated or unsaturated acyl residue with 8-24 carbon atoms and n the average number of ethylene oxide units per molecule, for numbers of 10-100, preferably 10-30 moles ethylene oxide to 1 mole caprylic acid, 2-ethylhexanoic acid, capric acid, lauric acid, isotridecanoic acid, myristic acid, cetylic acid, palmitoleic acid, stearic acid, isostearic acid, oleic acid, elaidic acid, petroselic acid, arachic acid, gadoleic acid, behenic acid, erucic acid and brassidic acid and technical-grade mixtures thereof. Adducts of 10-100 moles ethylene oxide to industrial fatty acids with 12-18 carbon atoms, such as coconut, palm, palm kernel or tallow fatty acid, are also suitable. Particularly preferred are PEG-50 monostearate, PEG-100 monostearate, PEG-50 monooleate, PEG-100 monooleate, PEG-50 monolaurate and PEG-100 monolaurate.

The C₁₂-C₁₈ alkanols or the C₁₂-C₁₈ carboxylic acids with in each case 10-30 units of ethylene oxide per molecule and mixtures of these substances, in particular ceteth-12, ceteth-20, ceteth-30, isoceteth-20, steareth-12, steareth-20, steareth-21, steareth-30, ceteareth-12, ceteareth-20, ceteareth-30, laureth-12 and beheneth-20, are particularly preferably used.

Moreover, C₈-C₂₂ alkyl mono- and oligoglycosides are preferably used. C₈-C₂₂ alkyl mono- and oligoglycosides represent known, commercial surfactants and emulsifiers. They are produced in particular by reacting glucose or oligosaccharides with primary alcohols having 8-22 carbon atoms. With respect to the glycoside residue, both monoglycosides in which a cyclic sugar residue is glycosidically bound to the fatty alcohol and oligomeric glycosides with a degree of oligomerization of up to about 8, preferably 1-2, are considered suitable. The degree of oligomerization here is a statistical average based on a homolog distribution which is conventional for those industrial products. Products which are available with the trade mark Plantacare® contain a glucosidically bound C₈-C₁₆ alkyl group on an oligoglucoside residue, the average degree of oligomerization of which is 1-2, in particular 1.1-1.4. Particularly preferred C₈-C₂₂ alkyl mono- and oligoglycosides are selected from octyl glucoside, decyl glucoside, lauryl glucoside, palmityl glucoside, isostearyl glucoside, stearyl glucoside, arachidyl glucoside and behenyl glucoside and mixtures thereof. The acyl glucamides derived from glucamine are also suitable as nonionic oil-in-water emulsifiers. Ethoxylated sterols, in particular ethoxylated soy sterols, also represent suitable oil-in-water emulsifiers according to the invention. The degree of ethoxylation must be greater than 5, preferably at least 10, in order to have an HLB value greater than 7. Suitable commercial products are e.g. PEG-10 soy sterol, PEG-16 soy sterol and PEG-25 soy sterol.

Moreover, partial esters of polyglycerols with 2 to 10 glycerol units and esterified with 1 to 4 saturated or unsaturated, linear or branched, optionally hydroxylated C₈-C₃₀ fatty acid residues are preferably used, provided that they have an HLB value of more than 7. Particularly preferred are diglycerol monocaprylate, diglycerol monocaprate, diglycerol monolaurate, triglycerol monocaprylate, triglycerol monocaprate, triglycerol monolaurate, tetraglycerol monocaprylate, tetraglycerol monocaprate, tetraglycerol monolaurate, pentaglycerol monocaprylate, pentaglycerol monocaprate, pentaglycerol monolaurate, hexaglycerol monocaprylate, hexaglycerol monocaprate, hexaglycerol monolaurate, hexaglycerol monomyristate, hexaglycerol monostearate, decaglycerol monocaprylate, decaglycerol monocaprate, decaglycerol monolaurate, decaglycerol monomyristate, decaglycerol monoisostearate, decaglycerol monostearate, decaglycerol monooleate, decaglycerol monohydroxystearate, decaglycerol dicaprylate, decaglycerol dicaprate, decaglycerol dilaurate, decaglycerol dimyristate, decaglycerol diisostearate, decaglycerol distearate, decaglycerol dioleate, decaglycerol dihydroxystearate, decaglycerol tricaprylate, decaglycerol tricaprate, decaglycerol trilaurate, decaglycerol trimyristate, decaglycerol triisostearate, decaglycerol tristearate, decaglycerol trioleate and decaglycerol trihydroxystearate.

Particularly preferred antiperspirant compositions according to the invention, in particular antiperspirant sticks, contain at least one active antiperspirant ingredient, at least 5 wt. % free water, 0.5-15 wt. % 1,4-dimethylolcyclohexane and at least one nonionic oil-in-water emulsifier in a total quantity of 0.5-10 wt. %, particularly preferably 1-4 wt. % and extraordinarily preferably 1.5-3 wt. %, based in each case on the overall composition.

### Water-in-Oil Emulsifiers

Preferred compositions according to the invention which are formulated as an emulsion, in particular as a water-in-oil emulsion, contain at least one water-in-oil emulsifier with an HLB value greater than 1.0 and less than or equal to 7.0, preferably at least one nonionic water-in-oil emulsifier with an HLB value greater than 1.0 and ≦7.0.

It may be preferred according to the invention to use only a single water-in-oil emulsifier. In another preferred embodiment, the compositions according to the invention contain mixtures, in particular technical-grade mixtures, of at least two water-in-oil emulsifiers.

Some of these suitable emulsifiers are listed e.g. in Kirk-Othmer, “Encyclopedia of Chemical Technology”, 3rd edition, 1979, volume 8, page 913. For ethoxylated adducts, the HLB value can also be calculated as already mentioned.

Preferred water-in-oil emulsifiers with an HLB value greater than 1.0 and 7.0 are selected from the mono- and diesters of ethylene glycol and the mono-, di-, tri- and tetraesters of pentaerythritol with linear, saturated fatty acids having 12-30, in particular 14-22 carbon atoms, which may be hydroxylated, and mixtures thereof. Preferred according to the invention are the mono- and diesters. Preferred C₁₂-C₃₀ fatty acid residues according to the invention are selected from lauric acid, myristic acid, palmitic acid, stearic acid, arachic acid and behenic acid residues; the stearic acid residue is particularly preferred. Particularly preferred nonionic water-in-oil emulsifiers according to the invention with an HLB value greater than 1.0 and 7.0 are selected from pentaerythrityl monostearate, pentaerythrityl distearate, pentaerythrityl tristearate, pentaerythrityl tetrastearate, ethylene glycol monostearate, ethylene glycol distearate and mixtures thereof. Particularly preferred water-in-oil emulsifiers according to the invention with an HLB value greater than 1.0 and 7.0 are available, for example, as commercial products Cutina® PES (INCI: Pentaerythrityl distearate), Cutina® AGS (INCI: Glycol distearate) or Cutina® EGMS (INCI: Glycol stearate). These commercial products already represent mixtures of mono- and diesters (in the case of the pentaerythrityl esters, tri- and tetraesters are also contained). A technical-grade mixture is understood to be e.g. a commercial product such as Cutina® PES.

Other preferred water-in-oil emulsifiers are:
linear, saturated alkanols with 12-30 carbon atoms, in particular with 16-22 carbon atoms, in particular cetyl alcohol, stearyl alcohol, arachidyl alcohol, behenyl alcohol and lanolin alcohol or mixtures of these alcohols, as obtainable from the industrial hydrogenation of plant and animal fatty acids,
esters and in particular partial esters of a polyol with 3-6 C atoms and linear, saturated and unsaturated fatty acids with 12-30, in particular 14-22 C atoms, which may be hydroxylated. These esters or partial esters are e.g. the mono- and diesters of glycerol or the monoesters of propylene glycol with linear, saturated and unsaturated C₁₂-C₃₀ carboxylic acids, which may be hydroxylated, in particular those with palmitic and stearic acid, the sorbitan mono-, di- or triesters of linear, saturated and unsaturated C₁₂-C₃₀ carboxylic acids, which may be hydroxylated, in particular those of myristic acid, palmitic acid, stearic acid or of mixtures of these fatty acids and the methyl glucose mono- and diesters of linear, saturated and unsaturated C₁₂-C₃₀ carboxylic acids, which may be hydroxylated;
sterols, i.e. steroids, which carry a hydroxyl group on the C3 atom of the steroid skeleton and are isolated both from animal tissue (zoosterols, e.g. cholesterol, lanosterol) and from plants (phytosterols, e.g. ergosterol, stigmasterol, sitosterol) and from fungi and yeasts (mycosterols) and can have a low degree of ethoxylation (1-5 EO);
alkanols and carboxylic acids with in each case 8-24 C atoms, in particular with 16-22 C atoms, in the alkyl group and 1-4 ethylene oxide units per molecule, having an HLB value greater than 1.0 and less than or equal to 7.0;
glycerol monoethers of saturated and/or unsaturated, branched and/or unbranched alcohols with a chain length of 8-30, in particular 12-18 carbon atoms;
partial esters of polyglycerols with n=2 to 10 glycerol units and esterified with 1 to 5 saturated or unsaturated, linear or branched, optionally hydroxylated C₈-C₃₀ fatty acid residues, provided that they have an HLB value of less than or equal to 7,
and mixtures of the aforementioned substances.

Water-in-oil emulsifiers which can be used particularly advantageously are stearyl alcohol, cetyl alcohol, glyceryl monostearate, glyceryl distearate, glyceryl monocaprate, glyceryl monocaprylate, glyceryl monolaurate, glyceryl monomyristate, glyceryl monopalmitate, glyceryl monohydroxystearate, glyceryl monooleate, glyceryl monolanolate, glyceryl dimyristate, glyceryl dipalmitate, glyceryl dioleate, propylene glycol monostearate, propylene glycol monolaurate, sorbitan monocaprylate, sorbitan monolaurate, sorbitan monomyristate, sorbitan monopalmitate, sorbitan monostearate, sorbitan sesquistearate, sorbitan distearate, sorbitan dioleate, sorbitan sesquioleate, sucrose distearate, arachidyl alcohol, behenyl alcohol, polyethylene glycol (2) stearyl ether (steareth-2), steareth-5, oleth-2, diglycerol monostearate, diglycerol monoisostearate, diglycerol monooleate, diglycerol dihydroxystearate, diglycerol distearate, diglycerol dioleate, triglycerol distearate, tetraglycerol monostearate, tetraglycerol distearate, tetraglycerol tristearate, decaglycerol pentastearate, decaglycerol pentahydroxystearate, decaglycerol pentaisostearate, decaglycerol pentaoleate, soy sterol, PEG-1 soy sterol, PEG-5 soy sterol, PEG-2 monolaurate and PEG-2 monostearate.

Other preferred W/O emulsifiers according to the invention are silicone-free polymeric water-in-oil emulsifiers, in particular PEG-30 dipolyhydroxystearate, available e.g. with the trade name Arlacel P 135 from Uniqema. With the aid of this emulsifier, in particular low-viscosity and even sprayable water-in-oil emulsions can be formulated.

Particularly preferred antiperspirant compositions according to the invention contain at least one active antiperspirant ingredient, at least 5 wt. % free water, 0.5-15 wt. % 1,4-dimethylolcyclohexane and at least one silicone-free water-in-oil emulsifier in a total quantity of 0.1-15 wt. %, preferably 0.5-8.0 wt. % and particularly preferably 1-4 wt. %, based in each case on the overall composition. Moreover, total quantities of at least one silicone-free water-in-oil emulsifier of 2-3-3.5 wt. %, based on the total weight of the composition, can be extraordinarily preferred according to the invention.

Other particularly preferred antiperspirant compositions according to the invention are present as a water-in-oil emulsion and contain at least one active antiperspirant ingredient, at least 5 wt. % free water, 0.5-15 wt. % 1,4-dimethylolcyclohexane and at least one silicone-free water-in-oil emulsifier in a total quantity of 0.1-15 wt. %, preferably 0.5-8.0 wt. % and particularly preferably 1-4 wt. %, based in each case on the overall composition. Moreover, in the aforementioned water-in-oil emulsions, total quantities of at least one silicone-free water-in-oil emulsifier of 2-3-3.5 wt. %, based on the total weight of the composition, can be extraordinarily preferred according to the invention.

Other preferred antiperspirant compositions according to the invention contain, based in each case on their total weight, at least one active antiperspirant ingredient, at least 5 wt. % free water, 0.5-15 wt. % 1,4-dimethylolcyclohexane and at least one polysaccharide. Other preferred antiperspirant compositions according to the invention are formulated as an oil-in-water emulsion which does not represent a microemulsion and contain, based in each case on their total weight, 0.5-15 wt. % 1,4-dimethylolcyclohexane, 0.5-6.5 wt. % oil or fat phase, encompassing at least one oil component which is liquid at 20° C. selected from linear and branched, saturated mono- or polyhydric C₃-C₃₀ alkanols which are etherified with at least one propylene oxide unit per molecule, propylene glycol monoesters of branched, saturated C₆-C₃₀ alkane carboxylic acids and branched, saturated C₁₀-C₃₀ alkanols, and also at least 60 wt. % free water and at least one active antiperspirant ingredient. Particularly preferred antiperspirant compositions of this type contain, based in each case on their total weight, 0.5-15 wt. % 1,4-dimethylolcyclohexane, 0.5-6.5 wt. % oil or fat phase, encompassing at least one oil component which is liquid at 20° C. selected from linear and branched, saturated mono- or polyhydric C₃-C₃₀ alkanols which are etherified with at least one propylene oxide unit per molecule, propylene glycol monoesters of branched, saturated C₆-C₃₀ alkane carboxylic acids and branched, saturated C₁₀-C₃₀ alkanols, and also at least 60 wt. % free water, at least one active antiperspirant ingredient and at least one polysaccharide and are suitable for application with a roller ball applicator.

Polysaccharides (glycans, polyglycans) is the collective name for macromolecular carbohydrates, the molecules of which consist of a large number (at least >10, but usually considerably more) of monosaccharide molecules (glycoses) glycosidically linked together.

The preferred polysaccharides according to the invention include especially the biopolymers starch, cellulose and dextran, which can be interpreted as a polycondensation product of D-glucose (polyglucosans, glucans), inulin as a polycondensate of D-fructose (polyfructosan, fructan), chitin and alginic acid.

Suitable polysaccharides according to the invention are understood to be both non-modified polysaccharides, such as e.g. xanthan or starch, and chemically modified polysaccharide derivatives, such as e.g. aluminum starch octenylsuccinate, hydroxypropyl methyl cellulose or dehydrated xanthan (INCI: Dehydroxanthan Gum), and physically modified polysaccharides, e.g. a starch pregelatinized by thermal treatment.

Preferred polysaccharides according to the invention are selected from starches, in particular from maize, potatoes and wheat, the components thereof, such as amylose and amylopectin, starch hydrolyzates and starch degradation products, such as maltodextrin, the physically or chemically modified starch derivatives, in particular the anionic starch derivatives aluminum starch octenylsuccinate, sodium starch octenylsuccinate, calcium starch octenylsuccinate, distarch phosphates, hydroxyethyl starch phosphates, hydroxypropyl starch phosphates, sodium carboxymethyl starches and sodium starch glycolate, cellulose, the chemically modified cellulose derivatives methyl cellulose, hydroxypropyl cellulose, hydroxyethyl cellulose, hydroxypropyl methyl cellulose, hydroxypropyl ethyl cellulose, hydroxyethyl methyl cellulose and carboxymethyl cellulose. Polysaccharides which form gums, such as e.g. guar gum, xanthan gum, dehydroxanthan gum, alginates, in particular sodium alginate, gum arabic, karaya gum, carrageenans, locust bean gum, linseed gums and agar-agar, can also be contained but are less preferred. In a particularly preferred embodiment, the compositions according to the invention are free from polysaccharide gums. In another particularly preferred embodiment, the compositions according to the invention are free from guar gum, xanthan gum, dehydroxanthan gum, alginates, in particular sodium alginate, gum arabic, karaya gum, carrageenans, locust bean gum, linseed gums and agar-agar.

Particularly preferred antiperspirant compositions according to the invention, in particular those in the form of oil-in-water emulsions, are characterized in that the at least one polysaccharide is selected from anionic and nonionic polysaccharides and mixtures thereof.

Other particularly preferred antiperspirant compositions according to the invention, in particular those in the form of oil-in-water emulsions, are characterized in that the at least one polysaccharide is selected from anionic and nonionic polysaccharides which do not form polysaccharide gums.

Other particularly preferred antiperspirant compositions according to the invention, in particular those in the form of oil-in-water emulsions, are characterized in that the anionic polysaccharide is selected from aluminum starch octenylsuccinate, sodium starch octenylsuccinate, calcium starch octenylsuccinate, distarch phosphates, hydroxyethyl starch phosphates, hydroxypropyl starch phosphates, sodium carboxymethyl starches, sodium starch glycolate and mixtures thereof. An extraordinarily preferred anionic polysaccharide according to the invention is aluminum starch octenylsuccinate.

Other particularly preferred antiperspirant compositions according to the invention, in particular those in the form of oil-in-water emulsions, are characterized in that the nonionic polysaccharide is selected from starches, starch hydrolyzates, cellulose, methyl cellulose, hydroxypropyl cellulose, hydroxyethyl cellulose, hydroxypropyl methyl cellulose, hydroxypropyl ethyl cellulose, hydroxyethyl methyl cellulose and mixtures thereof.

Other particularly preferred antiperspirant compositions according to the invention, in particular those in the form of oil-in-water emulsions, are characterized in that the at least one polysaccharide is contained in a total quantity of 0.01-1.0 wt. %, preferably 0.05-0.5 and particularly preferably 0.09-0.2 wt. %, based in each case on the total weight of the emulsion.

Preferred antiperspirant compositions according to the invention in the form of oil-in-water emulsions are characterized in that the at least one linear or branched, saturated mono- or polyhydric C₃-C₃₀ alkanol, which is etherified with at least one propylene oxide unit per molecule, is selected from addition products of at least 6 propylene oxide units per molecule to mono- or polyhydric C₃₋₃₀ alkanols, in particular to butanol, butanediol, myristyl alcohol and stearyl alcohol. Particularly preferred compounds of this type are selected from PPG-13 butyl ether, PPG-14 butyl ether, PPG-9 butyl ether, PPG-10 butanediol and PPG-15 stearyl ether and mixtures thereof.

Other preferred antiperspirant compositions according to the invention in the form of oil-in-water emulsions are characterized in that the at least one propylene glycol monoester of branched, saturated C₆-C₃₀ alkane carboxylic acids is selected from propylene glycol monoisostearate, propylene glycol monoisopalmitate, propylene glycol monoisobehenate, propylene glycol monoisoarachinate, propylene glycol monoisomyristate, propylene glycol monoisocaprate, propylene glycol monoisocaprinate and propylene glycol monoisocaprylate and mixtures thereof. Other preferred antiperspirant compositions according to the invention in the form of oil-in-water emulsions are characterized in that the at least one branched, saturated C₁₀-C₃₀ alkanol is selected from isostearyl alcohol, isocetyl alcohol, isomyristyl alcohol, isotridecyl alcohol, isoarachidyl alcohol, isobehenyl alcohol, isocapryl alcohol, isocaprinyl alcohol, isocaprylyl alcohol and mixtures thereof.

Other preferred antiperspirant compositions according to the invention in the form of oil-in-water emulsions are characterized in that at least one nonionic emulsifier with an HLB value in the range of 3-6 is contained. Particularly preferably, the at least one nonionic emulsifier with an HLB value in the range of 3-6 is selected from linear, saturated and unsaturated C₁₂-C₃₀ alkanols, which are etherified with 1-4 ethylene oxide units per molecule, which are extraordinarily preferably from steareth, ceteth, myristeth, laureth, trideceth, arachideth and beheneth with in each case 1-4 ethylene oxide units per molecule, in particular steareth-2, steareth-3, steareth-4, ceteth-2, ceteth-3, ceteth-4, myristeth-2, myristeth-3, myristeth-4, laureth-2, laureth-3, laureth-4, trideceth-2, trideceth-3 and trideceth-4 and mixtures thereof.

Other preferred antiperspirant compositions according to the invention in the form of oil-in-water emulsions are characterized in that at least one nonionic emulsifier with an HLB value in the range of 3-6 is contained in a total quantity of 1.8-3 wt. %, based on the total weight of the composition according to the invention. Other preferred antiperspirant compositions according to the invention in the form of oil-in-water emulsions are characterized in that at least one nonionic emulsifier with an HLB value in the range of 3-6, selected from steareth-2, steareth-3, steareth-4, ceteth-2, ceteth-3, ceteth-4, myristeth-2, myristeth-3, myristeth-4, laureth-2, laureth-3, laureth-4, trideceth-2, trideceth-3 and trideceth-4 and mixtures thereof, is contained in a total quantity of 1.8-3 wt. %, based on the total weight of the composition according to the invention.

Other preferred antiperspirant compositions according to the invention in the form of oil-in-water emulsions are characterized in that at least one nonionic emulsifier with an HLB value in the range of 12-18 is contained. Other preferred antiperspirant compositions according to the invention in the form of oil-in-water emulsions are characterized in that the at least one nonionic emulsifier with an HLB value in the range of 12-18 is selected from linear, saturated and unsaturated C₁₂-C₂₄ alkanols which are etherified with 7-40 ethylene oxide units per molecule. Particularly preferably, the aforementioned emulsifiers are selected from steareth, ceteth, myristeth, laureth, trideceth, arachideth and beheneth with in each case 7-40 ethylene oxide units per molecule, in particular steareth-10, steareth-20, steareth-21, steareth-30, steareth-40, ceteth-10, ceteth-20, ceteth-21, ceteth-30, ceteth-40, laureth-10, laureth-20, laureth-30, trideceth-10, trideceth-20 and trideceth-30 and mixtures thereof.

Other preferred antiperspirant compositions according to the invention in the form of oil-in-water emulsions are characterized in that at least one nonionic emulsifier with an HLB value in the range of 12-18, which is preferably selected from steareth-10, steareth-20, steareth-21, steareth-30, steareth-40, ceteth-10, ceteth-20, ceteth-21, ceteth-30, ceteth-40, laureth-10, laureth-20, laureth-30, trideceth-10, trideceth-20 and trideceth-30 and mixtures thereof, is contained in a total quantity of 1-2 wt. %, based on the total weight of the composition according to the invention.

Other preferred antiperspirant compositions according to the invention in the form of oil-in-water emulsions are characterized in that at least one nonionic emulsifier with an HLB value in the range of 3-6 and at least one nonionic emulsifier with an HLB value in the range of 12-18 are contained.

Other preferred antiperspirant compositions according to the invention in the form of oil-in-water emulsions are characterized in that at least one nonionic emulsifier with an HLB value in the range of 3-6 is contained in a total quantity of 1.8-3 wt. % and at least one nonionic emulsifier with an HLB value in the range of 12-18 is contained in a total quantity of 1-2 wt. %, the quantitative data being based in each case on the total weight of the composition according to the invention.

Other preferred antiperspirant compositions according to the invention in the form of oil-in-water emulsions are characterized in that steareth-2 is contained as a nonionic emulsifier with an HLB value in the range of 3-6 and at the same time steareth-21 is contained as a nonionic emulsifier with an HLB value in the range of 12-18.

Other preferred antiperspirant compositions according to the invention in the form of oil-in-water emulsions are characterized in that steareth-2, steareth-21 and PPG-15 stearyl ether are contained.

Other preferred antiperspirant compositions according to the invention in the form of oil-in-water emulsions are characterized in that at least one polysaccharide, selected from aluminum starch octenylsuccinate and distarch phosphates, is contained. Other preferred antiperspirant compositions according to the invention in the form of oil-in-water emulsions are characterized in that steareth-2, steareth-21, PPG-15 stearyl ether and aluminum starch octenylsuccinate are contained.

Other preferred antiperspirant compositions according to the invention in the form of oil-in-water emulsions are characterized in that the weight ratio of nonionic emulsifiers with an HLB value in the range of 3-6 and nonionic emulsifiers with an HLB value in the range of 12-18 is 0.9 to 3, preferably 1.3-1.9. Other preferred antiperspirant compositions according to the invention in the form of oil-in-water emulsions are characterized in that a total of no more than 3 wt. %, preferably no more than 1 wt. % and particularly preferably 0 wt. %, based in each case on the total weight of the composition according to the invention, of monohydric C₁-C₃ alkanols, such as ethanol or isopropanol, is contained.

Other preferred compositions according to the invention which are formulated as a water-in-oil emulsion contain at least one silicone-based water-in-oil emulsifier. Preferred antiperspirant compositions according to the invention contain at least one active antiperspirant ingredient, at least 5 wt. % free water, 0.5-15 wt. % 1,4-dimethylolcyclohexane and at least one silicone-based water-in-oil emulsifier in a total quantity of 0.1-5 wt. %, particularly preferably 0.5-3.5 wt. %, extraordinarily preferably 1.0-3 wt. %, more extraordinarily preferably 1.5-2.5 wt. %, based in each case on the total weight of the emulsion. Preferred antiperspirant compositions according to the invention in the form of a water-in-oil emulsion contain at least one active antiperspirant ingredient, at least 5 wt. % free water, 0.5-15 wt. % 1,4-dimethylolcyclohexane and at least one silicone-based water-in-oil emulsifier in a total quantity of 0.1-5 wt. %, particularly preferably 0.5-3.5 wt. %, extraordinarily preferably 1.0-3 wt. %, more extraordinarily preferably 1.5-2.5 wt. %, based in each case on the total weight of the emulsion.

A particularly preferred group of silicone-based water-in-oil emulsifiers according to the invention are the poly(C₂-C₃)alkylene glycol-modified silicones, which formerly had the INCI name Dimethicone Copolyol, with the current INCI names PEG-x Dimethicone (with x=2-20, preferably 3-17, particularly preferably 11-12), Bis-PEG-y Dimethicone (with y=3-25, preferably 4-20), PEG/PPG a/b Dimethicone (wherein a and b independently of one another denote numbers of 2-30, preferably 3-30 and particularly preferably 12-20, in particular 14-18), Bis-PEG/PPG-c/d Dimethicone (wherein c and d independently of one another denote numbers of 10-25, preferably 14-20 and particularly preferably 14-16) and Bis-PEG/PPG-e/f PEG/PPG g/h Dimethicone (wherein e, f, g and h independently of one another denote numbers of 10-20, preferably 14-18 and particularly preferably 16). Particularly preferred are PEG/PPG-18/18 Dimethicone, which is commercially available e.g. in a 1:9 mixture with cyclomethicone with the name Dow Corning 3225 C or Dow Corning 5225 C, PEG/PPG-18/18 Dimethicone, which is commercially available e.g. in a 1:3 mixture with non-volatile dimethicone as Dow Corning ES 5227 DM Formulation Aid, PEG/PPG-4/12 Dimethicone, which is available e.g. with the name Abil B 8852, and Bis-PEG/PPG-14/14 Dimethicone, which is commercially available e.g. in a mixture with cyclomethicone as Abil EM 97 (Evonik), Bis-PEG/PPG-20/20 Dimethicone, which is available e.g. with the name Abil B 8832, PEG/PPG-5/3 Trisiloxane (e.g. Silsoft 305) and PEG/PPG-20/23 Dimethicone (e.g. Silsoft 430 and Silsoft 440).

Other preferred silicone-based W/O emulsifiers according to the invention are poly(C₂-C₃)alkylene glycol-modified silicones, which are hydrophobically modified with C₄-C₁₈ alkyl groups, particularly preferably Cetyl PEG/PPG-10/1 Dimethicone (formerly: Cetyl Dimethicone Copolyol, e.g. available as Abil EM 90 or in a mixture of polyglyceryl-4 isostearate, Cetyl PEG/PPG-10/1 Dimethicone and hexyl laurate with the commercial name Abil WE 09), and also alkyl methicone copolyols and alkyl dimethicone ethoxy glucosides.

Particularly preferred antiperspirant compositions according to the invention contain at least one active antiperspirant ingredient, at least 5 wt. % free water, 0.5-15 wt. % 1,4-dimethylolcyclohexane and the silicone-based water-in-oil emulsifier PEG/PPG-18/18 Dimethicone in a total quantity of 0.5-5 wt. %, particularly preferably 1.0-3.5 wt. %, extraordinarily preferably 1.5-3 wt. %, based in each case on the total weight of the antiperspirant composition.

Other particularly preferred antiperspirant compositions according to the invention are present as a water-in-oil emulsion and contain at least one active antiperspirant ingredient, at least 5 wt. % free water, 0.5-15 wt. % 1,4-dimethylolcyclohexane and the silicone-based water-in-oil emulsifier PEG/PPG-18/18 Dimethicone in a total quantity of 0.5-5 wt. %, particularly preferably 1.0-3.5 wt. %, extraordinarily preferably 1.5-3 wt. %, based in each case on the total weight of the antiperspirant composition.

In the following table, various oil-in-water emulsifiers and water-in-oil-emulsifiers and their HLB values are compiled. However, the HLB values can also be calculated by Griffin's method, e.g. as illustrated or tabulated in ROMPP Chemie Lexikon, in particular in the online version of November 2003, and the handbooks of Fiedler, Kirk-Othmer and Janistyn cited there with the keyword “HLB System”. Where there are different figures in the literature for the HLB value of a substance, the HLB value which comes closest to the value calculated by Griffin's method should be used for the teaching according to the invention. If no clear HLB value can be established in this way, the HLB value given by the manufacturer of the emulsifier should be used for the teaching according to the invention. If this is also impossible, the HLB value should be determined experimentally.

HLB Value Chemical Name
(from H. Janistyn, Handbuch der Kosmetika and Riechstoffe, Hüthig-Verlag Heidelberg, 3^{rd} edition, 1978, volume 1, page 470 and volume 3, pages 68-78)

| | |
|---|---|
| 1 | Triglyceride saturated fatty acids |
| | Glyceryl trioleate |
| 1.5 | Ethylene glycol distearate |
| 1.6 | PurCellin oil |
| 1.8 | Sorbitan trioleate |
| | Glycerol dioleate |
| 2.1 | Sorbitan tristearate |
| 2.4 | Propylene glycol lactostearate |
| 2.7 | Glycerol monooleate |
| | Sorbitol dioleate |
| 2.8 | Glycerol monostearate |
| | Propylene glycol mono-/distearate, non-self-emulsifying |
| 2.9 | Ethylene glycol monostearate |
| 3.0 | Decaglycerol decaoleate |
| | Decaglycerol decastearate |
| | Generol 122 (Rapeseed Sterols) |
| | Sucrose distearate |
| 3.1 | Decaglycerol decaoleate |
| | Glyceryl monoricinoleate |
| | Pentaerythrityl monostearate |
| | Pentaerythrityl sesquioleate |
| 3.2 | Ethylene glycol monodistearate, non-self-emulsifying |
| | Glycol stearate |
| 3.3 | Glycerol monolaurate |
| 3.4 | Propylene glycol monostearate |
| 3.5 | Ethylene glycol monostearate |
| | Pentaerythrityl monooleate |
| | Polyethylene glycol (100) monooleate |
| 3.6 | Glycerol mono-/dioleate, non-self-emulsifying |
| | Monoethoxy lauryl ether |
| 3.7 | Sorbitan sesquioleate (Dehymuls SSO) |
| 3.8 | Glycerol monodistearate, non-self-emulsifying |
| | Polyethylene glycol (100) monostearate |
| | Diglycerol sesquioleate |
| | N,N-Dimethyl caproamide |
| | Pentaerythritol monotallate |
| | Propylene glycol monolaurate |
| 4.0 | Decaglycerol octaoleate |
| 4.3 | Sorbitan monooleate (Dehymuls SMO) |
| | Diethylene glycol monostearate |
| 4.4 | 1,2-Propylene glycol monodistearate, self-emulsifying |
| 4.5 | Glycerol monostearate palmitate (90%), non-self-emulsifying |
| | Propylene glycol monolaurate |
| 4.7 | Sorbitan monostearate (Dehymuls SMS) |
| | Diethylene glycol monooleate |
| 4.8 | Pentaerythritol monolaurate |
| 4.9 | Polyoxyethylene (2) oleyl alcohol (polyoxyethylene (2) oleyl ether) |
| | Polyoxyethylene (2) stearyl alcohol (polyoxyethylene (2) stearyl ether) |
| 5.0 | Ethylene glycol monodistearate |
| | Generol 122 E 5 (PEG-5 soy sterol) |
| | Polyethylene glycol (100) monoricinoleate |
| | Polyethylene glycol (200) distearate |
| | Polyglyceryl-3 isostearate (e.g. Isolan Gl 34 from Tego) |
| 5.9 | Polyethylene glycol (200) dilaurate |
| 6.0 | Decaglycerol tetraoleate |
| | Polyethylene glycol (100) monolaurate |
| | Polyethylene glycol (200) dioleate |
| 6.1 | Diethylene glycol monolaurate (diglycol laurate) |
| 6.3 | Polyethylene glycol (300) dilaurate |
| 6.4 | Glycerol monoricinoleate |
| | Glycerol sorbitan monolaurate |
| 6.5 | Diethylene glycol monolaurate |
| | Sodium stearoyl-2 lactylate |
| 6.7 | Sorbitan monopalmitate |
| 6.8 | Glycerol monococoate |
| | Glycerol monolaurate |
| 7.0 | Polyoxyethylene (2) C₁₀-C₁₄ fatty alcohol ether, laureth-2 (Dehydol LS 2) |
| | Sucrose distearate |
| 7.2 | Polyethylene glycol (400) dioleate |
| | Sucrose dioleate |
| 7.4 | Polyethylene glycol (100) monolaurate |
| | Sucrose dipalmitate |
| 7.5 | Sucrose dipalmitate |
| 7.6 | Glycerol sorbitan laurate |
| 7.8 | Polyethylene glycol (400) distearate |
| 7.9 | Polyethylene glycol (200) monostearate |
| | Polyoxyethylene (3) tridecyl alcohol |
| 8-8.2 | Polyethylene glycol (400) distearate |
| 8.0 | Polyoxyethylene (3) C₁₀-C₁₄ fatty alcohol ether, laureth-3 (Dehydol LS 3) |
| | N,N-dimethyllauramide |
| | Sodium lauroyl lactylate, sodium lauroyl-2 lactylate |
| | Polyethylene glycol (200) monooleate |
| | Polyethylene glycol (220) monotallate |
| | Polyethylene glycol (1500) dioleate |
| | Polyoxyethylene (4) oleyl alcohol |
| | Polyoxyethylene (4) stearyl cetyl ether |
| 8.2 | Triglycerol monooleate |
| 8.3 | Diethylene glycol monolaurate |
| 8.4 | Polyoxyethylene (4) cetyl ether |
| | Polyoxyethylene glycol (400) dioleate |
| 8.5 | Sodium caproyl lactylate |
| | Polyethylene glycol (200) monostearate |
| | Sorbitan monooleate |
| 8.6 | Sorbitan monolaurate (Dehymuls SML) |
| | Polyethylene glycol (200) monolaurate |
| 8.8 | Polyoxyethylene (4) myristyl ether |
| | Polyethylene glycol (400) dioleate |
| 8.9 | Nonylphenol, polyethoxylated with 4 moles EO |
| 9.0 | Oleth-5 (e.g. Eumulgin O 5) |
| 9.2-9.7 | Polyoxyethylene (4) lauryl alcohol (depending on commercial product, |
| | e.g. Brij 30, Dehydol LS 4) |
| 9.3 | Polyoxyethylene (4) tridecyl alcohol |
| 9.6 | Polyoxyethylene (4) sorbitan monostearate |
| 9.8 | Polyethylene glycol (200) monolaurate |
| 10-11 | Polyethylene glycol (400) monooleate |
| 10.0 | Didodecyl dimethyl ammonium chloride |
| 10.0 | Polyethylene glycol (200) monolaurate |
| | Polyethylene glycol (400) dilaurate |
| | Polyethylene glycol (600) dioleate |
| | Polyoxyethylene (4) sorbitan monostearate |
| | Polyoxyethylene (5) sorbitan monooleate |
| 10.2 | Polyoxyethylene (40) sorbitol hexaoleate |
| 10.4-10.6 | Polyoxyethylene glycol (600) distearate |
| 10.5 | Polyoxyethylene (20) sorbitan tristearate |
| 10.6 | Sucrose monostearate |
| 10.7 | Sucrose monooleate |
| 11-11.4 | Polyethylene glycol (400) monooleate |
| 11.0 | Polyethylene glycol (350) monostearate |
| | Polyethylene glycol (400) monotallate |
| | Polyoxyethylene glycol (7) monostearate |
| | Polyoxyethylene glycol (8) monooleate |
| | Polyoxyethylene (20) sorbitan trioleate |
| | Polyoxyethylene (6) tridecyl alcohol |
| 11.1 | Polyethylene glycol (400) monostearate |
| 11.2 | Polyoxyethylene (9) monostearate |
| | Sucrose monooleate |
| | Sucrose monostearate |
| 11.4 | Polyoxyethylene (50) sorbitol hexaoleate |
| | Sucrose monotallate |
| | Sucrose stearate palmitate |
| 11.6 | Polyoxyethylene glycol (400) monoricinoleate |
| 11.7 | Sucrose monomyristate |
| | Sucrose monopalmitate |
| 12.0 | PEG-10 soy sterol (e.g. Generol 122 E 10) |
| | Triethanolamine oleate |
| 12.2-12.3 | Nonylphenol, ethoxylated with 8 moles EO |
| 12.2 | Sucrose monomyristate |
| 12.4 | Sucrose monolaurate |
| | Polyoxyethylene (10) oleyl alcohol, polyoxyethylene (10) oleyl ether |
| | Polyoxyethylene (10) stearyl alcohol, polyoxyethylene (10) stearyl ether |
| 12.5 | Polyoxyethylene (10) stearyl cetyl ether |
| 12.7 | Polyoxyethylene (8) tridecyl alcohol |
| 12.8 | Polyoxyethylene glycol (400) monolaurate |
| | Sucrose monococoate |
| 12.9 | Polyoxyethylene (10) cetyl ether |
| 13 | Glycerol monostearate, ethoxylated (20 moles EO) |
| 13.0 | Eumulgin O 10 |
| | Eumulgin 286 |
| | Eumulgin B 1 (ceteareth-12) |
| 13.0 | C12 fatty amines, ethoxylated (5 moles EO) |
| 13.1 | Nonylphenol, ethoxylated (9.5 moles EO) |
| 13.2 | Polyethylene glycol (600) monostearate |
| | Polyoxyethylene (16) tall oil |
| 13.3 | Polyoxyethylene (4) sorbitan monolaurate |
| 13.5 | Nonylphenol, ethoxylated (10.5 moles EO) |
| | Polyethylene glycol (600) monooleate |
| 13.7 | Polyoxyethylene (10) tridecyl alcohol |
| | Polyethylene glycol (660) monotallate |
| | Polyethylene glycol (1500) monostearate |
| | Polyoxyethylene glycol (1500) dioleate |
| 13.9 | Polyethylene glycol (400) monococoate |
| | Polyoxyethylene (9) monolaurate |
| 14-16 | Eumulgin HRE 40 (castor oil, ethoxylated with 40 EO and hydrogenated) |
| 14.0 | Polyoxyethylene (12) lauryl ether |
| | Polyoxyethylene (12) tridecyl alcohol |
| 14.2 | Polyoxyethylene (15) stearyl alcohol |
| 14.3 | Polyoxyethylene (15) stearyl cetyl ether |
| 14.4 | Mixture of C12-C15 fatty alcohols with 12 moles EO |
| 14.5 | Polyoxyethylene (12) lauryl alcohol |
| 14.8 | Polyoxyethylene glycol (600) monolaurate |
| 14.9-15.2 | Sorbitan monostearate, ethoxylated with 20 EO (e.g. Eumulgin SMS 20) |
| 15-15.9 | Sorbitan monooleate, ethoxylated with 20 EO (e.g. Eumulgin SMO 20) |
| 15.0 | PEG-20 Glyceryl stearate (e.g. Cutina E 24) |
| | PEG-40 Castor Oil (e.g. Eumulgin RO 40) |
| | Decyl glucoside (Oramix NS 10) |
| | Dodecyl glucoside (Plantaren APG 600) |
| | Dodecyltrimethylammonium chloride |
| | Nonylphenol, ethoxylated with 15 moles EO |
| | Polyethylene glycol (1000) monostearate |
| | Polyoxyethylene (600) monooleate |
| 15-17 | Eumulgin HRE 60 (castor oil, ethoxylated with 60 EO and hydrogenated) |
| 15.3 | C12 fatty amines, polyethoxylated with 12 moles EO |
| | Polyoxyethylene (20) oleyl alcohol, Polyoxyethylene (20) oleyl ether |
| 15.4 | Polyoxyethylene (20) stearyl cetyl ether (e.g. Eumulgin B 2 (ceteareth- |
| | 20)) |
| 15.5 | Polyoxyethylene (20) stearyl alcohol |
| 15.6 | Polyoxyethylene glycol (1000) monostearate |
| | Polyoxyethylene (20) sorbitan monopalmitate |
| 15.7 | Polyoxyethylene (20) cetyl ether |
| 15.9 | Disodium triethanolamine distearyl heptaglycol ether sulfosuccinate |
| 16.0 | Nonylphenol ethoxylated with 20 moles EO |
| | Polyoxyethylene (25) propylene glycol stearate |
| 16-16.8 | Polyoxyethylene (30) monostearate |
| 16.3-16.9 | Polyoxyethylene (40) monostearate |
| 16.5-16.7 | Polyoxyethylene (20) sorbitan monolaurate (e.g. Eumulgin SML 20) |
| 16.6 | Polyoxyethylene (20) sorbitol |
| 16.7 | C18 fatty amines, polyethoxylated with 5 moles EO |
| | Polyoxyethylene (23) lauryl alcohol |
| 17.0 | Ceteareth-30, e.g. Eumulgin B 3 |
| | Octyl glucoside (Triton CG 110) |
| | Polyoxyethylene (30) glyceryl monolaurate |
| 17.1 | Nonylphenol, ethoxylated with 30 moles EO |
| 17.4 | Polyoxyethylene (40) stearyl alcohol |

Other preferred compositions according to the invention are characterized in that the total content of nonionic and ionic emulsifiers and/or surfactants with an HLB value greater than 8 is no more than 20 wt. %, preferably no more than 15 wt. %, particularly preferably no more than 10 wt. %, particularly preferably no more than 7 wt. %, also particularly preferably no more than 4 wt. % and extraordinarily preferably no more than 3 wt. %, based in each case on the total composition according to the invention.

### Oils

Preferred compositions according to the invention which are present as an emulsion preferably also contain at least one oil which is liquid at 20° C., which is not an odorant component or essential oil. Preferred oils according to the invention are selected from branched, saturated or unsaturated fatty alcohols with 6-30 carbon atoms. These alcohols are often also referred to as Guerbet alcohols, since they are obtainable by the Guerbet reaction. Preferred alcohol oils are hexyl decanol, octyl dodecanol and 2-ethylhexyl alcohol. Other preferred oil components are mixtures of Guerbet alcohols and Guerbet alcohol esters, preferably mixtures of hexyl decanol and hexyldecyl laurate, which are available e.g. as the commercial product Cetiol® PGL.

Other preferred oils according to the invention are selected from the triglycerides of linear or branched, saturated or unsaturated, optionally hydroxylated C₈₋₃₀ fatty acids. The use of natural oils, e.g. soybean oil, cottonseed oil, sunflower seed oil, palm oil, palm kernel oil, linseed oil, almond oil, castor oil, corn oil, olive oil, rapeseed oil, sesame oil, safflower oil, wheat germ oil, peach kernel oil and the liquid fractions of coconut oil and the like can be particularly suitable. However, synthetic triglyceride oils with unbranched fatty acid residues, in particular capric/caprylic triglycerides, and synthetic triglyceride oils with branched fatty acid residues, in particular glyceryl triisostearin, are also suitable.

Other particularly preferred oils according to the invention are selected from the dicarboxylic acid esters of linear or branched C₂-C₁₀ alkanols, in particular diisopropyl adipate, di-n-butyl adipate, di-(2-ethylhexyl)adipate, dioctyl adipate, diethyl/di-n-butyl/dioctyl sebacate, diisopropyl sebacate, dioctyl malate, dioctyl maleate, dicaprylyl maleate, diisooctyl succinate, di-2-ethylhexyl succinate and di-(2-hexyldecyl)succinate.

Other particularly preferred oils according to the invention are selected from the addition products of 1 to 5 propylene oxide units to mono- or polyhydric C₈₋₂₂ alkanols such as octanol, decanol, decanediol, lauryl alcohol, myristyl alcohol and stearyl alcohol, e.g. PPG-2 myristyl ether and PPG-3 myristyl ether.

Other preferred oil components according to the invention are selected from the esters of linear or branched, saturated or unsaturated fatty alcohols having 2-30 carbon atoms with linear or branched, saturated or unsaturated fatty acids having 2-30 carbon atoms, which may be hydroxylated. Preferred oils of this type are hexyldecyl stearate, hexyldecyl laurate, isodecyl neopentanoate, isononyl isononanoate, 2-ethylhexyl palmitate and 2-ethylhexyl stearate. Other preferred oil components according to the invention are isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid 2-butyloctanoate, diisotridecyl acetate, n-butyl stearate, n-hexyl laurate, n-decyl oleate, oleyl oleate, oleyl erucate, erucyl oleate, erucyl erucate, ethylene glycol dioleate and ethylene glycol dipalmitate.

Other preferred oil components according to the invention are selected from the addition products of at least 6 ethylene oxide and/or propylene oxide units to mono- or polyhydric C₃₋₂₂ alkanols such as butanol, butanediol, myristyl alcohol and stearyl alcohol, e.g. PPG-14 butyl ether, PPG-9 butyl ether, PPG-10 butanediol and PPG-15 stearyl ether.

Other preferred oil components according to the invention are selected from the C₈-C₂₂ fatty alcohol esters of monovalent or polyvalent C₂-C₇ hydroxycarboxylic acids, in particular the esters of glycolic acid, lactic acid, malic acid, tartaric acid, citric acid and salicylic acid. Those esters based on linear C_{14/15} alkanols, e.g. C₁₂-C₁₅ alkyl lactate, and on C_{12/13} alkanols branched in the 2-position can be purchased with the trade mark Cosmacol® from Nordmann, Rassmann GmbH & Co, Hamburg, in particular the commercial products Cosmacol® ESI, Cosmacol® EMI and Cosmacol® ETI.

Other preferred oil components according to the invention are selected from the symmetrical, asymmetrical or cyclic esters of carbonic acid with fatty alcohols, e.g. dicaprylyl carbonate or the esters of DE 197 56 454 A1.

Other preferred oil components according to the invention are selected from the esters of dimers of unsaturated C₁₂-C₂₂ fatty acids (dimer fatty acids) with monohydric linear, branched or cyclic C₂-C₁₈ alkanols or with polyhydric linear or branched C₂-C₆ alkanols.

It can be extraordinarily preferred according to the invention to use mixtures of the aforementioned oils.

Other preferred oil components according to the invention are selected from silicone oils and hydrocarbon oils.

Preferred silicone oils according to the invention are selected from dialkyl and alkylaryl siloxanes, which include e.g. dimethyl polysiloxane and methyl phenyl polysiloxane, but also hexamethyl disiloxane, octamethyl trisiloxane and decamethyl tetrasiloxane. Cosmetic oils are differentiated into volatile and non-volatile oils. Non-volatile oils are understood to be those oils that have a vapor pressure of less than 2.66 Pa (0.02 mm Hg) at 20° C. and an ambient pressure of 1013 hPa. Volatile oils are understood to be those oils that have a vapor pressure of 2.66 Pa-40000 Pa (0.02 mm-300 mm Hg), preferably 13-12000 Pa (0.1-90 mm Hg), particularly preferably 15-8000 Pa, extraordinarily preferably 300-3000 Pa, at 20° C. and an ambient pressure of 1013 hPa.

Volatile cosmetic oils are generally selected from among cyclic silicone oils with the INCI name Cyclomethicone. The INCI name Cyclomethicone is understood in particular to be cyclotrisiloxane(hexamethylcyclotrisiloxane), cyclotetrasiloxane(octamethylcyclotetrasiloxane), cyclopentasiloxane(decamethylcyclopentasiloxane) and cyclohexasiloxane(dodecamethyl-cyclohexasiloxane). These oils have a vapor pressure of approx. 13-15 Pa at 20° C.

Other preferred silicone oils according to the invention are selected from volatile silicone oils, which can be cyclic, such as e.g. octamethylcyclotetrasiloxane, decamethylcyclopentasiloxane and dodecamethylcyclohexasiloxane and mixtures thereof, as are contained e.g. in the commercial products DC 244, 245, 344 and 345 from Dow Corning, or linear, e.g. hexamethyldisiloxane (L₂), octamethyltrisiloxane (L₃), decamethyltetrasiloxane (L₄), dodecamethylpentasiloxane (L₅), and any two-, three- and four-component mixtures of L₂, L₃, L₄ and/or L₅, as are contained e.g. in the commercial products Dow Corning 2-1184 Fluid, Dow Corning 200 (0.65 cSt) and Dow Corning® 200 (1.5 cSt) from Dow Corning, the values of the kinematic viscosity referring to a temperature of 25° C.

Other preferred silicone oils according to the invention are selected from non-volatile, higher molecular weight, linear dimethylpolysiloxanes, commercially available e.g. with the name Dow Corning® 190, Dow Corning® 200 Fluid with kinematic viscosities in the range of 5-100 cSt, preferably 5-50 cSt or also 5-10 cSt, and Baysilon® 350 M, the values of the kinematic viscosity referring to a temperature of 25° C.

Preferred natural and synthetic hydrocarbons according to the invention are selected from isoalkanes, C₈-C₁₆ isoalkanes, C₁₀-C₁₃ isoalkanes, paraffin oils, isohexadecane, isoeicosane, polyisobutenes, hydrogenated polyisobutenes and polydecenes, which are available e.g. with the name Emery® 3004, 3006, 3010 or with the name Ethylflo® from Albemarle or Nexbase® 2004G from Nestle, and 1,3-di-(2-ethylhexyl)cyclohexane.

Particularly preferred antiperspirant compositions according to the invention are characterized in that the oil/s which is/are liquid at 20° C. is/are contained in a total quantity of 0.1-80 wt. %, preferably 2-20 wt. %, particularly preferably 3-15 wt. %, based in each case on the total weight of the composition.

In another preferred embodiment of the invention, a proportion of the oil components of at least 80 wt. % has a refractive index n_{D} of 1.39-1.51. It is particularly preferred if 5-40-50 wt. %, extraordinarily preferably 10-12-25-30 wt. % of the oil components have a refractive index n_{D} of 1.43-1.58, preferably 1.44-1.51, particularly preferably 1.45-1.47-1.49, at 20° C. (measured at λ=589 nm).

Other particularly preferred antiperspirant compositions according to the invention are present as a water-in-oil emulsion in gel form and contain at least one active antiperspirant ingredient, at least 5 wt. % free water, 0.5-15 wt. % 1,4-dimethylolcyclohexane, at least one water-in-oil emulsifier, in particular a silicone-based water-in-oil emulsifier, and 1-40 wt. % of at least one cosmetic oil, the quantitative data being based in each case on the total weight of the antiperspirant composition. In a preferred embodiment, these water-in-oil emulsion gels have a dynamic viscosity at 25° C. in the range of 15,000 to 300,000 mPas, preferably 30,000 to 150,000 mPas and extraordinarily preferably 40,000 to 100,000 mPas. In another preferred embodiment, the water-in-oil emulsion gels according to the invention with a dynamic viscosity at 25° C. in the range of 15,000 to 300,000 mPas, preferably 30,000 to 150,000 mPas and extraordinarily preferably 40,000 to 100,000 mPas are transparent. Compositions with a haze of no more than 100 NTU, preferably no more than 70 NTU, particularly preferably no more than 50 NTU, measured in each case at 25° C., are considered transparent. The transparency is preferably achieved by adjusting the refractive index of the aqueous phase and the oil phase to the same value before mixing. For particularly high transparency, a matching of the refractive indices to ±0.001, preferably to ±0.0005 or better is required.

In another preferred embodiment, the water-in-oil emulsions according to the invention are present in stick form.

Other particularly preferred antiperspirant compositions according to the invention are present as a water-in-oil emulsion and contain at least one active antiperspirant ingredient, at least 5 wt. % free water, 0.5-15 wt. % 1,4-dimethylolcyclohexane, at least one water-in-oil emulsifier, in particular a silicone-based water-in-oil emulsifier, and 1-40 wt. % of at least one cosmetic oil, the quantitative data being based in each case on the total weight of the propellant-free antiperspirant composition, and are packed with at least one propellant in an aerosol dispensing device.

Surprisingly, it has been found that water-in-oil emulsions according to the invention have particularly high temperature stability. It has also been found that water-in-oil emulsions according to the invention which are packed with a propellant in an aerosol dispensing device exhibit particularly low corrosiveness.

Water-in-oil emulsion sprays contain no zirconium-containing salts because of legal provisions. The zirconium-free active antiperspirant ingredients described above are also suitable for water-in-oil emulsion sprays according to the invention. Preferred compositions according to the invention which are formulated as a water-in-oil emulsion spray contain at least one zirconium-free aluminum salt as active antiperspirant ingredient in a total quantity of 5-40 wt. %, preferably 10-35 wt. % and particularly preferably 15-28 wt. % and extraordinarily preferably 23-27 wt. %, based in each case on the total weight of the active substance (USP) free from water of crystallization in the propellant-free overall composition.

Preferred compositions according to the invention, which are formulated as a water-in-oil emulsion gel, stick or spray, contain free water in a total quantity of 10-85 wt. %, preferably 15-75 wt. %, particularly preferably 20-65 wt. %, extraordinarily preferably 25-55 wt. %, also extraordinarily preferably 30-40 wt. %, based in each case on the total weight of the propellant-free overall composition.

The external phase of water-in-oil emulsions according to the invention encompasses at least one cosmetic oil. Oils which are preferred as a component of preferred water-in-oil emulsions according to the invention have already been listed above. Preferred water-in-oil emulsions according to the invention contain at least one active antiperspirant ingredient, 10-85 wt. % free water, 0.5-15 wt. % 1,4-dimethylolcyclohexane, at least one water-in-oil emulsifier, in particular a silicone-based water-in-oil emulsifier, and 1-40 wt. % of at least one cosmetic oil selected from branched, saturated or unsaturated fatty alcohols with 6-30 carbon atoms, dicarboxylic acid esters of linear or branched C₂-C₁₀ alkanols, addition products of 1 to 5 propylene oxide units to mono- or polyhydric C₈₋₂₂ alkanols, addition products of at least 6 ethylene oxide and/or propylene oxide units to mono- or polyhydric C₃₋₂₂ alkanols, esters of linear or branched, saturated or unsaturated fatty alcohols having 2-30 carbon atoms with linear or branched, saturated or unsaturated fatty acids having 2-30 carbon atoms, which may be hydroxylated, dicaprylyl carbonate, octamethylcyclotetrasiloxane, decamethylcyclopentasiloxane, dodecamethylcyclohexasiloxane, hexamethyldisiloxane (L₂), octamethyltrisiloxane (L₃), decamethyltetrasiloxane (L₄), dodecamethylpentasiloxane (L₅) and any two-, three- and four-component mixtures of L₂, L₃, L₄ and/or L₅, linear dimethylpolysiloxanes with kinematic viscosities in the range of 5-100 cSt, the values of the kinematic viscosity referring to a temperature of 25° C., C₈-C₁₆ isoalkanes, C₁₀-C₁₃ isoalkanes, isohexadecane, isoeicosane, polyisobutenes, hydrogenated polyisobutenes and polydecenes and mixtures of the aforementioned oils, the quantitative data being based in each case on the total weight of the (propellant-free) antiperspirant composition.

Owing to their particularly favorable residue properties, water-in-oil emulsions are preferred according to the invention which, based in each case on their total weight, contain at least one active antiperspirant ingredient, 10-85 wt. % free water, 0.5-15 wt. % 1,4-dimethylolcyclohexane, at least one water-in-oil emulsifier, in particular a silicone-based water-in-oil emulsifier, and 1-40 wt. % of at least one cosmetic oil selected from two- and three-component mixtures of C₈-C₁₆ isoalkanes, linear dimethylpolysiloxanes with kinematic viscosities in the range of 5-100 cSt (25° C.) and esters of linear or branched, saturated or unsaturated fatty alcohols having 2-30 carbon atoms with linear or branched, saturated or unsaturated fatty acids having 2-30 carbon atoms, which may be hydroxylated.

Particularly preferred are water-in-oil emulsions which, based in each case on their total weight, contain at least one active antiperspirant ingredient, 10-85 wt. % free water, 0.5-15 wt. % 1,4-dimethylolcyclohexane, at least one water-in-oil emulsifier, in particular a silicone-based water-in-oil emulsifier, and 1-40 wt. % of at least one cosmetic oil selected from two- and three-component mixtures of C₈-C₁₆ isoalkanes, linear dimethylpolysiloxanes with kinematic viscosities in the range of 5-100 cSt (25° C.) and esters selected from hexyldecyl stearate, hexyldecyl laurate, isodecyl neopentanoate, isononyl isononanoate, 2-ethylhexyl palmitate, 2-ethylhexyl stearate, isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid 2-butyloctanoate, diisotridecyl acetate, n-butyl stearate and n-hexyl laurate.

Moreover, water-in-oil emulsions are preferred which contain at least one active antiperspirant ingredient, 10-85 wt. % free water, 0.5-15 wt. % 1,4-dimethylolcyclohexane, at least one water-in-oil emulsifier, in particular a silicone-based water-in-oil emulsifier, and 1-40 wt. % of at least one cosmetic oil selected from two- and three-component mixtures of C₈-C₁₆ isoalkanes, linear dimethylpolysiloxanes with kinematic viscosities in the range of 5-100 cSt (25° C.) and esters of linear or branched, saturated or unsaturated fatty alcohols having 2-30 carbon atoms with linear or branched, saturated or unsaturated fatty acids having 2-30 carbon atoms, which may be hydroxylated, and are packed with at least one propellant in an aerosol dispensing device, the quantitative data being based in each case on the total weight of the propellant-free antiperspirant composition.

Particularly preferred are water-in-oil emulsions which contain at least one active antiperspirant ingredient, 10-85 wt. % free water, 0.5-15 wt. % 1,4-dimethylolcyclohexane, at least one water-in-oil emulsifier, in particular a silicone-based water-in-oil emulsifier, and 1-40 wt. % of at least one cosmetic oil selected from two- and three-component mixtures of C₈-C₁₆ isoalkanes, linear dimethylpolysiloxanes with kinematic viscosities in the range of 5-100 cSt (25° C.) and esters selected from hexyldecyl stearate, hexyldecyl laurate, isodecyl neopentanoate, isononyl isononanoate, 2-ethylhexyl palmitate, 2-ethylhexyl stearate, isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid 2-butyloctanoate, diisotridecyl acetate, n-butyl stearate and n-hexyl laurate, and are packed with at least one propellant in an aerosol dispensing device, the quantitative data being based in each case on the total weight of the propellant-free antiperspirant composition.

The at least one cosmetic oil is contained in the compositions according to the invention, which are present as an 0/W or W/O emulsion, in a total quantity of 1-40 wt. %, preferably 2-30 wt. %, particularly preferably 5-25 wt. % and extraordinarily preferably 10-20 wt. %, based in each case on the total weight of the (propellant-free) antiperspirant composition.

Other preferred water-in-oil emulsions according to the invention contain at least one active antiperspirant ingredient, 10-85 wt. % free water, 0.5-15 wt. % 1,4-dimethylolcyclohexane, at least one silicone-free water-in-oil emulsifier as described above in a total quantity of 0.1-15 wt. %, preferably 0.5-8.0 wt. % and particularly preferably 1-4 wt. %, and 1-40 wt. % of at least one cosmetic oil selected from branched, saturated or unsaturated fatty alcohols with 6-30 carbon atoms, dicarboxylic acid esters of linear or branched C₂-C₁₀ alkanols, addition products of 1 to 5 propylene oxide units to mono- or polyhydric C₈₋₂₂ alkanols, addition products of at least 6 ethylene oxide and/or propylene oxide units to mono- or polyhydric C₃₋₂₂ alkanols, esters of linear or branched, saturated or unsaturated fatty alcohols having 2-30 carbon atoms with linear or branched, saturated or unsaturated fatty acids having 2-30 carbon atoms, which may be hydroxylated, dicaprylyl carbonate, octamethylcyclotetrasiloxane, decamethylcyclopentasiloxane, dodecamethylcyclohexasiloxane, hexamethyldisiloxane (L₂), octamethyltrisiloxane (L₃), decamethyltetrasiloxane (L₄), dodecamethylpentasiloxane (L₅), and any two-, three- and four-component mixtures of L₂, L₃, L₄ and/or L₅, linear dimethylpolysiloxanes with kinematic viscosities in the range of 5-100 cSt, the values of the kinematic viscosity referring to a temperature of 25° C., C₈-C₁₆ isoalkanes, C₁₀-C₁₃ isoalkanes, isohexadecane, isoeicosane, polyisobutenes, hydrogenated polyisobutenes and polydecenes and mixtures of the aforementioned oils, the quantitative data being based in each case on the total weight of the (propellant-free) antiperspirant composition. In another preferred embodiment, these water-in-oil emulsions are packed with at least one propellant in an aerosol dispensing device.

Other preferred water-in-oil emulsions according to the invention contain at least one active antiperspirant ingredient, 10-85 wt. % free water, 0.5-15 wt. % 1,4-dimethylolcyclohexane, at least one silicone-based water-in-oil emulsifier as described above in a total quantity of 0.1-5 wt. %, particularly preferably 0.5-3.5 wt. %, extraordinarily preferably 1.0-3 wt. %, and 1-40 wt. % of at least one cosmetic oil selected from branched, saturated or unsaturated fatty alcohols with 6-30 carbon atoms, dicarboxylic acid esters of linear or branched C₂-C₁₀ alkanols, addition products of 1 to 5 propylene oxide units to mono- or polyhydric C₈₋₂₂ alkanols, addition products of at least 6 ethylene oxide and/or propylene oxide units to mono- or polyhydric C₃₋₂₂ alkanols, esters of linear or branched, saturated or unsaturated fatty alcohols having 2-30 carbon atoms with linear or branched, saturated or unsaturated fatty acids having 2-30 carbon atoms, which may be hydroxylated, dicaprylyl carbonate, octamethylcyclotetrasiloxane, decamethylcyclopentasiloxane, dodecamethylcyclohexasiloxane, hexamethyldisiloxane (L₂), octamethyltrisiloxane (L₃), decamethyltetrasiloxane (L₄), dodecamethylpentasiloxane (L₅) and any two-, three- and four-component mixtures of L₂, L₃, L₄ and/or L₅, linear dimethylpolysiloxanes with kinematic viscosities in the range of 5-100 cSt, the values of the kinematic viscosity referring to a temperature of 25° C., C₈-C₁₆ isoalkanes, C₁₀-C₁₃ isoalkanes, isohexadecane, isoeicosane, polyisobutenes, hydrogenated polyisobutenes and polydecenes and mixtures of the aforementioned oils, the quantitative data being based in each case on the total weight of the (propellant-free) antiperspirant composition. In another preferred embodiment, these water-in-oil emulsions are packed with at least one propellant in an aerosol dispensing device.

Other preferred water-in-oil emulsions according to the invention contain at least one active antiperspirant ingredient, 10-85 wt. % free water, 0.5-15 wt. % 1,4-dimethylolcyclohexane, at least one silicone-based water-in-oil emulsifier, selected PEG/PPG-18/18 dimethicone, in a total quantity of 0.1-5 wt. %, particularly preferably 0.5-3.5 wt. %, extraordinarily preferably 1.0-3 wt. %, and 1-40 wt. % of at least one cosmetic oil selected from branched, saturated or unsaturated fatty alcohols with 6-30 carbon atoms, dicarboxylic acid esters of linear or branched C₂-C₁₀ alkanols, addition products of 1 to 5 propylene oxide units to mono- or polyhydric C₈₋₂₂ alkanols, addition products of at least 6 ethylene oxide and/or propylene oxide units to mono- or polyhydric C₃₋₂₂ alkanols, esters of linear or branched, saturated or unsaturated fatty alcohols having 2-30 carbon atoms with linear or branched, saturated or unsaturated fatty acids having 2-30 carbon atoms, which may be hydroxylated, dicaprylyl carbonate, octamethylcyclotetrasiloxane, decamethylcyclopentasiloxane, dodecamethylcyclohexasiloxane, hexamethyldisiloxane (L₂), octamethyltrisiloxane (L₃), decamethyltetrasiloxane (L₄), dodecamethylpentasiloxane (L₅) and any two-, three- and four-component mixtures of L₂, L₃, L₄ and/or L₅, linear dimethylpolysiloxanes with kinematic viscosities in the range of 5-100 cSt, the values of the kinematic viscosity referring to a temperature of 25° C., C₈-C₁₆ isoalkanes, C₁₀-C₁₃ isoalkanes, isohexadecane, isoeicosane, polyisobutenes, hydrogenated polyisobutenes and polydecenes and mixtures of the aforementioned oils, the quantitative data being based in each case on the total weight of the (propellant-free) antiperspirant composition. In another preferred embodiment, these water-in-oil emulsions are packed with at least one propellant in an aerosol dispensing device.

Other preferred water-in-oil emulsions according to the invention contain at least one active antiperspirant ingredient, 10-85 wt. % free water, 0.5-15 wt. % 1,4-dimethylolcyclohexane, at least one silicone-free water-in-oil emulsifier as described above in a total quantity of 0.1-15 wt. %, preferably 0.5-8.0 wt. %, and particularly preferably 1-4 wt. %, and 1-40 wt. % of at least one cosmetic oil selected from two- and three-component mixtures of C₈-C₁₆ isoalkanes, linear dimethylpolysiloxanes with kinematic viscosities in the range of 5-100 cSt (25° C.) and esters selected from hexyldecyl stearate, hexyldecyl laurate, isodecyl neopentanoate, isononyl isononanoate, 2-ethylhexyl palmitate, 2-ethylhexyl stearate, isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid 2-butyloctanoate, diisotridecyl acetate, n-butyl stearate and n-hexyl laurate, the quantitative data being based in each case on the total weight of the (propellant-free) antiperspirant composition. In another preferred embodiment, these water-in-oil emulsions are packed with at least one propellant in an aerosol dispensing device.

Other preferred water-in-oil emulsions according to the invention contain at least one active antiperspirant ingredient, 10-85 wt. % free water, 0.5-15 wt. % 1,4-dimethylolcyclohexane, at least one silicone-based water-in-oil emulsifier as described above in a total quantity of 0.1-5 wt. %, particularly preferably 0.5-3.5 wt. %, extraordinarily preferably 1.0-3 wt. %, and 1-40 wt. % of at least one cosmetic oil selected from two- and three-component mixtures of C₈-C₁₆ isoalkanes, linear dimethylpolysiloxanes with kinematic viscosities in the range of 5-100 cSt (25° C.) and esters selected from hexyldecyl stearate, hexyldecyl laurate, isodecyl neopentanoate, isononyl isononanoate, 2-ethylhexyl palmitate, 2-ethylhexyl stearate, isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid 2-butyloctanoate, diisotridecyl acetate, n-butyl stearate and n-hexyl laurate, the quantitative data being based in each case on the total weight of the (propellant-free) antiperspirant composition. In another preferred embodiment, these water-in-oil emulsions are packed with at least one propellant in an aerosol dispensing device.

Other preferred water-in-oil emulsions according to the invention contain at least one active antiperspirant ingredient, 10-85 wt. % free water, 0.5-15 wt. % 1,4-dimethylolcyclohexane, at least one silicone-based water-in-oil emulsifier selected from PEG/PPG-18/18 dimethicone, in a total quantity of 0.1-5 wt. %, particularly preferably 0.5-3.5 wt. %, extraordinarily preferably 1.0-3 wt. %, and 1-40 wt. % of at least one cosmetic oil selected from two- and three-component mixtures of C₈-C₁₆ isoalkanes, linear dimethylpolysiloxanes with kinematic viscosities in the range of 5-100 cSt (25° C.) and esters selected from hexyldecyl stearate, hexyldecyl laurate, isodecyl neopentanoate, isononyl isononanoate, 2-ethylhexyl palmitate, 2-ethylhexyl stearate, isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid 2-butyloctanoate, diisotridecyl acetate, n-butyl stearate and n-hexyl laurate, the quantitative data being based in each case on the total weight of the (propellant-free) antiperspirant composition. In another preferred embodiment, these water-in-oil emulsions are packed with at least one propellant in an aerosol dispensing device.

In another preferred embodiment, the preferred water-in-oil emulsions according to the invention contain at least one active antiperspirant ingredient, 10-85 wt. % free water, 0.5-15 wt. % 1,4-dimethylolcyclohexane, at least one silicone-free water-in-oil emulsifier as described above in a total quantity of 0.1-15 wt. %, preferably 0.5-8.0 wt. %, and particularly preferably 1-4 wt. %, 1-40 wt. % of at least one cosmetic oil and at least one nonionic polyalkylene glycol ether with an HLB value >7, which is preferably selected from isoceteth-20, the quantitative data being based in each case on the total weight of the (propellant-free) antiperspirant composition. The nonionic polyalkylene glycol ether with an HLB value >7 is preferably contained in a total quantity of 0.1-3 wt. %, preferably 0.5-1.5 wt. %, based in each case on the total weight of the (propellant-free) antiperspirant composition. In another preferred embodiment, these water-in-oil emulsions are packed with at least one propellant in an aerosol dispensing device.

In another preferred embodiment, the preferred water-in-oil emulsions according to the invention contain at least one active antiperspirant ingredient, 10-85 wt. % free water, 0.5-15 wt. % 1,4-dimethylolcyclohexane, at least one silicone-based water-in-oil emulsifier as described above in a total quantity of 0.1-5 wt. %, particularly preferably 0.5-3.5 wt. %, extraordinarily preferably 1.0-3 wt. %, 1-40 wt. % of at least one cosmetic oil and at least one nonionic polyalkylene glycol ether with an HLB value >7, which is preferably selected from isoceteth-20, the quantitative data being based in each case on the total weight of the (propellant-free) antiperspirant composition. The nonionic polyalkylene glycol ether with an HLB value >7 is preferably contained in a total quantity of 0.1-3 wt. %, preferably 0.5-1.5 wt. %, based in each case on the total weight of the (propellant-free) antiperspirant composition. In another preferred embodiment, these water-in-oil emulsions are packed with at least one propellant in an aerosol dispensing device.

In another preferred embodiment, the preferred water-in-oil emulsions according to the invention contain at least one active antiperspirant ingredient, 10-85 wt. % free water, 0.5-15 wt. % 1,4-dimethylolcyclohexane, at least one silicone-based water-in-oil emulsifier selected from PEG/PPG-18/18 dimethicone in a total quantity of 0.1-5 wt. %, particularly preferably 0.5-3.5 wt. %, extraordinarily preferably 1.0-3 wt. %, 1-40 wt. % of at least one cosmetic oil and at least one nonionic polyalkylene glycol ether with an HLB value >7, which is preferably selected from isoceteth-20, the quantitative data being based in each case on the total weight of the (propellant-free) antiperspirant composition. The nonionic polyalkylene glycol ether with an HLB value >7 is preferably contained in a total quantity of 0.1-3 wt. %, preferably 0.5-1.5 wt. %, based in each case on the total weight of the (propellant-free) antiperspirant composition. In another preferred embodiment, these water-in-oil emulsions are packed with at least one propellant in an aerosol dispensing device.

Other preferred water-in-oil emulsions according to the invention contain at least one active antiperspirant ingredient, 10-85 wt. % free water, 0.5-15 wt. % 1,4-dimethylolcyclohexane, at least one silicone-based water-in-oil emulsifier as described above in a total quantity of 0.1-5 wt. %, particularly preferably 0.5-3.5 wt. %, extraordinarily preferably 1.0-3 wt. %, 1-40 wt. % of at least one cosmetic oil selected from two- and three-component mixtures of C₈-C₁₆ isoalkanes, linear dimethylpolysiloxanes with kinematic viscosities in the range of 5-100 cSt (25° C.) and esters selected from hexyldecyl stearate, hexyldecyl laurate, isodecyl neopentanoate, isononyl isononanoate, 2-ethylhexyl palmitate, 2-ethylhexyl stearate, isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid 2-butyloctanoate, diisotridecyl acetate, n-butyl stearate and n-hexyl laurate, and at least one nonionic polyalkylene glycol ether with an HLB value >7, which is preferably selected from isoceteth-20, the quantitative data being based in each case on the total weight of the (propellant-free) antiperspirant composition. The nonionic polyalkylene glycol ether with an HLB value >7 is preferably contained in a total quantity of 0.1-3 wt. %, preferably 0.5-1.5 wt. %, based in each case on the total weight of the (propellant-free) antiperspirant composition. In another preferred embodiment, these water-in-oil emulsions are packed with at least one propellant in an aerosol dispensing device.

Other preferred water-in-oil emulsions according to the invention contain at least one active antiperspirant ingredient, 10-85 wt. % free water, 0.5-15 wt. % 1,4-dimethylolcyclohexane, at least one silicone-based water-in-oil emulsifier selected from PEG/PPG-18/18 dimethicone in a total quantity of 0.1-5 wt. %, particularly preferably 0.5-3.5 wt. %, extraordinarily preferably 1.0-3 wt. %, 1-40 wt. % of at least one cosmetic oil selected from two- and three-component mixtures of C₈-C₁₆ isoalkanes, linear dimethylpolysiloxanes with kinematic viscosities in the range of 5-100 cSt (25° C.) and esters selected from hexyldecyl stearate, hexyldecyl laurate, isodecyl neopentanoate, isononyl isononanoate, 2-ethylhexyl palmitate, 2-ethylhexyl stearate, isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid 2-butyloctanoate, diisotridecyl acetate, n-butyl stearate and n-hexyl laurate, and at least one nonionic polyalkylene glycol ether with an HLB value >7, which is preferably selected from isoceteth-20, the quantitative data being based in each case on the total weight of the (propellant-free) antiperspirant composition. The nonionic polyalkylene glycol ether with an HLB value >7 is preferably contained in a total quantity of 0.1-3 wt. %, preferably 0.5-1.5 wt. %, based in each case on the total weight of the (propellant-free) antiperspirant composition. In another preferred embodiment, these water-in-oil emulsions are packed with at least one propellant in an aerosol dispensing device.

Surprisingly, it has been found that emulsions of this type exhibit particularly high temperature stability and particularly low corrosiveness.

Preferred compositions according to the invention contain, in addition to 1,4-dimethylolcyclohexane, at least one water-soluble polyhydric C₂-C₉ alkanol with 2-6 hydroxyl groups and/or at least one water-soluble polyethylene glycol with 3-20 ethylene oxide units and mixtures thereof in order to improve the stability of the compositions further. These components are preferably selected from 1,2-propylene glycol, 2-methyl-1,3-propanediol, glycerol, 1,2-butylene glycol, 1,3-butylene glycol, 1,4-butylene glycol, pentylene glycols, such as 1,2-pentanediol and 1,5-pentanediol, hexanediols, such as 1,6-hexanediol, hexanetriols, such as 1,2,6-hexanetriol, 1,2-octanediol, 1,8-octanediol, dipropylene glycol, tripropylene glycol, diglycerol, triglycerol, erythritol, sorbitol and mixtures of the aforementioned substances. Suitable water-soluble polyethylene glycols are selected from PEG-3, PEG-4, PEG-6, PEG-7, PEG-8, PEG-9, PEG-10, PEG-12, PEG-14, PEG-16, PEG-18 and PEG-20 and mixtures thereof, with PEG-3 to PEG-8 being preferred.

Particularly preferred antiperspirant compositions according to the invention are characterized in that the at least one water-soluble polyhydric C₂-C₉ alkanol with 2-6 hydroxyl groups and/or at least one water-soluble polyethylene glycol with 3-20 ethylene oxide units is contained in total quantities of 3-30 wt. %, preferably 8-25 wt. %, particularly preferably 10-18 wt. %, based in each case on the overall composition, the content of 1,4-dimethylolcyclohexane not being included here.

In particular the water-in-oil emulsions described above preferably contain at least one water-soluble polyhydric C₂-C₉ alkanol with 2-6 hydroxyl groups and/or at least one water-soluble polyethylene glycol with 3-20 ethylene oxide units in total quantities of 3-30 wt. %, preferably 8-25 wt. %, particularly preferably 10-18 wt. %, based in each case on the overall composition, the content of 1,4-dimethylolcyclohexane not being included here. These water-in-oil emulsions are particularly preferably packed with a propellant in an aerosol dispensing device.

Other preferred compositions according to the invention contain at least one hydrogel former. Compositions of this type exhibit surprisingly improved perfume adhesion and improved temperature stability as a result of 1,4-dimethylolcyclohexane. Preferred hydrogel formers are selected from cellulose ethers, especially hydroxyalkyl celluloses, in particular hydroxypropyl cellulose, hydroxypropyl methyl cellulose, hydroxyethyl cellulose, carboxymethyl cellulose, cetyl hydroxyethyl cellulose, hydroxybutyl methyl cellulose, methyl hydroxyethyl cellulose, and also xanthan gum, sclerotium gum, succinoglucans, polygalactomannans, in particular guar gums and locust bean gum, in particular guar gum and locust bean gum themselves and the nonionic hydroxyalkyl guar derivatives and locust bean gum derivatives, such as hydroxypropyl guar, carboxymethyl hydroxypropyl guar, hydroxypropyl methyl guar, hydroxyethyl guar and carboxymethyl guar, and also pectins, agar, carragheen (carrageenan), tragacanth, gum arabic, karaya gum, tara gum, gellan, gelatin, casein, pectin, propylene glycol alginate, alginic acids and salts thereof, in particular sodium alginate, potassium alginate and calcium alginate, and also polyvinylpyrrolidones, polyvinyl alcohols, polyacrylamides, and also physically (e.g. by pre-gelatinization) and/or chemically modified starches, in particular hydroxypropylated starch phosphates and octenyl starch succinates and aluminum, calcium or sodium salts thereof, and also water-soluble acrylic acid-acrylate copolymers, acrylic acid-acrylamide copolymers, acrylic acid-vinylpyrrolidone copolymers, acrylic acid-vinyl formamide copolymers and polyacrylates.

Preferred compositions according to the invention contain at least one hydrogel former in a total quantity of 0.1-3.0 wt. %, preferably 0.3-2.0 wt. %, particularly preferably 0.5-1.5 wt. % and extraordinarily preferably 0.7-1.0 wt. %, based in each case on the total weight of the overall composition according to the invention.

In another preferred embodiment, the compositions according to the invention take the form of a hydro gel and contain at least one hydrogel former in a total quantity of 0.1-3.0 wt. %, preferably 0.3-2.0 wt. %, particularly preferably 0.5-1.5 wt. % and extraordinarily preferably 0.7-1.0 wt. %, based in each case on the total weight of the overall composition according to the invention.

Particularly preferred compositions according to the invention also preferably contain at least one skin-cooling active ingredient. Those compounds which, like l-menthol, stimulate the heat receptors in the skin and the mucous membranes in such a way that a cool sensory impression results are regarded according to the invention as skin-cooling active ingredients. In particular the CMR-1 receptor (“cold- and menthol-sensitive receptor”), which belongs to the TRP family of channels, is stimulated by the active cooling ingredients, resulting in the production of an impression of cold. Suitable skin-cooling active ingredients according to the invention are e.g. menthol, isopulegol and menthol derivatives, e.g. menthyl lactate, menthyl glycolate, menthyl pyrrolidone carboxylic acid, menthyl methyl ether, menthoxypropanediol, menthone glycerol acetal (9-methyl-6-(1-methylethyl)-1,4-dioxaspiro (4.5) decane-2-methanol), monomenthyl succinate and 2-hydroxymethyl-3,5,5-trimethylcyclohexanol. Preferred as skin-cooling active ingredients are menthol, isopulegol, menthyl lactate, menthoxypropanediol and menthyl pyrrolidone carboxylic acid and mixtures of these substances, in particular mixtures of menthol and menthyl lactate, menthol, menthol glycolate and menthyl lactate, menthol and menthoxypropanediol or menthol and isopulegol.

It is particularly preferred according to the invention that at least one skin-cooling active ingredient is contained in a total quantity of 0.01-1 wt. %, particularly preferably 0.02-0.5 wt. % and extraordinarily preferably 0.05-0.2-0.3 wt. %, based in each case on the total weight of the composition.

Particularly preferred compositions according to the invention which are formulated as a propellant-driven aerosol contain at least one propellant. In particular the water-in-oil emulsions described above are preferably packed with a propellant in an aerosol dispensing device. Preferred propellants (propellant gases) are propane, propene, n-butane, isobutane, isobutene, n-pentane, pentene, isopentane, isopentene, methane, ethane, dimethyl ether, nitrogen, air, oxygen, laughing gas, 1,1,1,3-tetrafluorethane, heptafluoro-n-propane, perfluoro ethane, monochlorodifluoromethane and 1,1-difluoroethane (INCI: Hydrofluorocarbon 152a), both individually and in combination. Hydrophilic propellants, such as e.g. carbon dioxide, can also be used advantageously within the meaning of the present invention if a small proportion of hydrophilic gases is selected and lipophilic propellant (e.g. propane/butane) is present in excess. Particularly preferred are propane, n-butane, isobutane and mixtures of these propellants. It has been shown that the use of n-butane as the sole propellant can be particularly preferred according to the invention.

The quantity of propellant is preferably 20-80-90 wt. %, particularly preferably 30-70-75 wt. % and extraordinarily preferably 40-50 wt. %, based in each case on the total weight of the preparation consisting of the composition according to the invention and the propellant.

As compressed gas container, vessels made of metal (aluminum, tinplate, tin), protected or non-shattering plastics or glass which is externally coated with plastics are suitable, with pressure resistance and breaking strength, corrosion resistance, ease of filling and esthetic aspects, handling, printability etc. playing a part in the selection thereof. Special internal protective lacquers ensure corrosion resistance with respect to the composition according to the invention. The compositions according to the invention which are sprayed with a propellant can either be packed directly with the propellant or packed in a multi-chamber container in which the composition according to the invention is physically separated from the propellant.

### Low Melting Point Lipid or Wax Component

Particularly preferred antiperspirant compositions according to the invention, in particular those in stick form but also those present in gel form which are suitable for application in a roll-on, are characterized in that at least one wax component with a melting point in the range of 25 to <50° C. is contained, selected from coconut fatty acid glycerol mono-, di- and triesters, Butyrospermum parkii (shea butter) and esters of saturated, monohydric C₈-C₁₈ alcohols with saturated C₂-C₁₈ monocarboxylic acids and mixtures of these substances. These lower melting point wax components make it possible to optimize the consistency of products in stick or cream form or flowable products and to minimize visible residues on the skin. Particularly preferred are commercial products with the INCI name Cocoglycerides, in particular the commercial products Novata® (ex Cognis), particularly preferably Novata® AB, a mixture of C₂-C₁₈ mono-, di- and triglycerides, which melts in the range of 30-32° C., and the Softisan series of products (Sasol Germany GmbH) with the INCI name Hydrogenated Cocoglycerides, in particular Softisan 100, 133, 134, 138, 142. Other preferred esters of saturated, monohydric C₁₂-C₁₈ alcohols with saturated C₁₂-C₁₈ monocarboxylic acids are stearyl laurate, cetearyl stearate (e.g. Crodamol® CSS), cetyl palmitate (e.g. Cutina® CP) and myristyl myristate (e.g. Cetiol® MM).

Other particularly preferred antiperspirant compositions according to the invention, in particular those in stick form, are characterized in that the at least one wax component with a melting point in the range of 25-<50° C. is contained in quantities of 0.01 to 20 wt. %, preferably 0.1-10 wt. %, particularly preferably 0.5-5 wt. % and extraordinarily preferably 2-4 wt. %, based on the overall composition.

### Fillers

Particularly preferred antiperspirant compositions according to the invention are characterized in that, to improve their consistency and sensory properties, they also contain at least one solid, water-insoluble, particulate filler. In an extraordinarily preferred embodiment, this filler is selected from optionally modified starches (e.g. from maize, rice, potatoes) and starch derivatives, which are optionally pre-gelatinized, in particular starch derivatives such as aluminum starch octenylsuccinate (e.g. DRY FLO®), sodium starch octenylsuccinate, cellulose and cellulose derivatives, silicon dioxide, silicas, e.g. Aerosil® grades, spherical polyalkyl sesquisiloxane particles (in particular Aerosil® 8972 and Aerosil® 200V from Degussa), silica gels, talcum, kaolin, clays, e.g. bentonites, magnesium aluminum silicates, boron nitride, lactoglobulin derivatives, e.g. sodium C₈₋₁₆ isoalkylsuccinyl lactoglobulin sulfonate, glass powders, polymer powders, in particular from polyolefins, polycarbonates, polyurethanes, polyamides, e.g. nylon, polyesters, polystyrenes, polyacrylates, (meth)acrylate or (meth)acrylate-vinylidene copolymers, which may be crosslinked, or silicones, and mixtures of these substances.

Polymer powders based on a polymethacrylate copolymer are available e.g. as the commercial product Polytrap® 6603 (Dow Corning). Other polymer powders, e.g. based on polyamides, are available with the name Orgasol® 1002 (polyamide-6) and Orgasol® 2002 (polyamide-12) from Elf Atochem. Other polymer powders which are suitable for the purpose according to the invention are e.g. polymethacrylates (Micropearl® M from SEPPIC or Plastic Powder A from NIKKOL), styrene-divinylbenzene copolymers (Plastic Powder FP from NIKKOL), polyethylene and polypropylene powders (ACCUREL® EP 400 from AKZO) or silicone polymers (Silicone Powder X2-1605 from Dow Corning).

Particularly preferred antiperspirant compositions according to the invention are characterized in that they contain at least one solid, water-insoluble particulate filler in a total quantity of 0.01 to 30 wt. %, preferably 5-20 wt. %, particularly preferably 8-15 wt. %, based in each case on the overall composition.

### Odorants

Surprisingly, it has been found, that a content of 0.5-15 wt. % 1,4-dimethylolcyclohexane in the antiperspirant compositions according to the invention leads to prolonged perfume adhesion.

Preferred antiperspirant compositions according to the invention are therefore characterized in that at least one active antiperspirant ingredient, at least 5 wt. % free water, 0.5-15 wt. % 1,4-dimethylolcyclohexane and at least one odorant are contained.

The definition of an odorant within the meaning of the present application coincides with the definition which is conventional in the art, as can be taken from the ROMPP Chemie Lexikon as at December 2007. According to this, an odorant is a chemical compound with odor and/or taste, which stimulates the receptors of the hair cells (adequate stimulus). The physical and chemical properties required for this are a low molar mass of no more than 300 g/mol, high vapor pressure, minimal water solubility as well as high lipid solubility and weak polarity and the presence of at least one osmophoric group in the molecule. To differentiate volatile, low molecular weight substances which are not regarded and used as odorants, either conventionally or within the meaning of the present application, but primarily as solvents, such as e.g. ethanol, propanol, isopropanol and acetone, from odorants according to the invention, odorants according to the invention have a molar mass of 74 to 300 g/mol, contain at least one osmophoric group in the molecule and have an odor and/or taste, i.e. they stimulate the receptors of the hair cells of the olfactory system.

Perfumes, perfume oils, components of perfume oils or individual odorant compounds can be used as odorants. Perfume oils or perfumes according to the invention can be individual odorant compounds, e.g. the synthetic products of the type of the esters, ethers, aldehydes, ketones, alcohols and hydrocarbons. Odorant compounds of the type of the esters are e.g. benzyl acetate, phenoxyethyl isobutyrate, p-tert.-butylcyclohexyl acetate, linalyl acetate, dimethyl benzyl carbinyl acetate (DMBCA), phenylethyl acetate, benzyl acetate, ethyl methyl phenyl glycinate, allyl cyclohexyl propionate, styrallyl propionate, benzyl salicylate, cyclohexyl salicylate, Floramat, Melusat and Jasmecyclat. The ethers include e.g. benzyl ethyl ether and ambroxan, the aldehydes e.g. the linear alkanals with 8-18 C atoms, citral, citronellal, citronellal oxyacetaldehyde, cyclamen aldehyde, lilial and bourgeonal, the ketones e.g. the ionones, alpha-isomethyl ionone and methyl cedryl ketone, the alcohols anethole, citronellol, eugenol, geraniol, linalool, phenyl ethyl alcohol, alpha-terpineol, beta-terpineol, gamma-terpineol and delta-terpineol, and the hydrocarbons mainly include the terpenes, such as limonene and pinene. Preferably, however, mixtures of various odorants are used, which together produce an attractive fragrance note.

These perfume oils can also contain natural odorant mixtures, as can be obtained from plant sources, e.g. pine, citrus, jasmine, patchouli, rose or ylang-ylang oil. Also suitable are clary sage oil, chamomile oil, clove oil, melissa oil, mint oil, cinnamon leaf oil, linden blossom oil, juniper berry oil, vetiver oil, olibanum oil, galbanum oil and labdanum oil, as well as orange blossom oil, neroli oil, orange peel oil and sandalwood oil.

In order to be perceptible, an odorant must be volatile, the molar mass playing an important part in this together with the nature of the functional groups and the structure of the chemical compound. Thus, most odorants have molar masses of up to about 200 daltons while molar masses of 300 daltons and above are more of an exception. Owing to the different volatility of odorants, the odor of a perfume or odorant composed of several odorants changes during evaporation, the odor impressions being categorized as “top note”, “middle note or body” and “end note or dry out”. Since odor perception is also largely based on the odor intensity, the top note of a perfume or odorant does not consist only of volatile compounds, while the end note largely consists of less volatile, i.e. tenacious odorants. In the composition of perfumes, more volatile odorants can, for example, be bound to certain fixatives, thus preventing them from evaporating too rapidly. The following categorization of odorants into “more volatile” or “tenacious” odorants therefore gives no information on the odor impression or on whether the corresponding odorant is perceived as a top or middle note.

Tenacious perfumes which can be used within the framework of the present invention are e.g. the essential oils, such as angelica root oil, anise oil, arnica flower oil, basil oil, bay oil, bergamot oil, champaca flower oil, silver fir oil, silver fir cone oil, elemi oil, eucalyptus oil, fennel oil, spruce needle oil, galbanum oil, geranium oil, ginger grass oil, guaiacwood oil, gurjun balsam oil, helichrysum oil, ho oil, ginger oil, iris oil, cajeput oil, calamus oil, chamomile oil, camphor oil, cananga oil, cardamom oil, cassia oil, pine needle oil, copaiba balsam oil, coriander oil, spearmint oil, caraway oil, cumin oil, lavender oil, lemongrass oil, lime oil, mandarin oil, melissa oil, musk seed oil, myrrh oil, clove oil, neroli oil, niaouli oil, olibanum oil, orange oil, oregano oil, palmarosa oil, patchouli oil, Peru balsam oil, petitgrain oil, pepper oil, peppermint oil, pimento oil, pine oil, rose oil, rosemary oil, sandalwood oil, celery oil, spike oil, star anise oil, turpentine oil, thuja oil, thyme oil, verbena oil, vetiver oil, juniper berry oil, wormwood oil, wintergreen oil, ylang-ylang oil, hyssop oil, cinnamon oil, cinnamon leaf oil, citronella oil, lemon oil and cypress oil. However, the higher-boiling or solid odorants of natural or synthetic origin can also be used within the framework of the present invention as tenacious odorants or odorant mixtures, i.e. odorants. These compounds include the compounds mentioned below and mixtures thereof: ambrettolide, allyl acetate, alpha-amyl cinnamaldehyde, anethole, anisaldehyde, anisyl alcohol, anisole, anthranilic acid methyl ester, acetophenone, benzyl acetone, benzaldehyde, benzoic acid ethyl ester, benzophenone, benzyl alcohol, benzyl acetate, benzyl benzoate, benzyl formate, benzyl valerianate, borneol, bornyl acetate, α-bromostyrene, n-decyl aldehyde, n-dodecyl aldehyde, eugenol, eugenol methyl ether, eucalyptol, farnesol, fenchone, fenchyl acetate, geranyl acetate, geranyl formate, heliotropin, heptyne carboxylic acid methyl ester, heptaldehyde, hydroquinone dimethyl ether, hydroxycinnamaldehyde, hydroxycinnamyl alcohol, indole, irone, isoeugenol, isoeugenol methyl ether, isosafrole, jasmone, camphor, carvacrol, carvone, p-cresol methyl ether, coumarin, p-methoxyacetophenone, methyl-n-amyl ketone, methylanthranilic acid methyl ester, p-methyl acetophenone, methyl chavicol, p-methyl quinoline, methyl-β-naphthyl ketone, methyl-n-nonyl acetaldehyde, methyl-n-nonyl ketone, muscone, beta-naphthol ethyl ether, beta-naphthol methyl ether, nerol, nitrobenzene, n-nonyl aldehyde, nonyl alcohol, n-octyl aldehyde, p-oxy-acetophenone, pentadecanolide, beta-phenyl ethyl alcohol, phenylacetaldehyde dimethyl acetal, phenylacetic acid, pulegone, safrole, salicylic acid isoamyl ester, salicylic acid methyl ester, salicylic acid hexyl ester, salicylic acid cyclohexyl ester, santalol, skatole, alpha-terpineol, beta-terpineol, gamma-terpineol, delta-terpineol, thymene, thymol, gamma-undecalactone, vanillin, veratrum aldehyde, cinnamaldehyde, cinnamyl alcohol, cinnamic acid, cinnamic acid ethyl ester, cinnamic acid benzyl ester.

The more volatile odorants include in particular the lower boiling odorants of natural or synthetic origin, which can be used individually or in mixtures. Examples of more volatile odorants are alkyl isothiocyanates (alkyl mustard oils), butanedione, limonene, linalool, linalyl acetate, linalyl propionate, menthol, menthone, methyl-n-heptenone, phellandrene, phenylacetaldehyde, terpinyl acetate, citral and citronellal.

Particularly preferred antiperspirant compositions according to the invention are characterized in that at least one perfume component is contained in a total quantity of 0.00001 to 10 wt. %, preferably 0.5-7 wt. %, particularly preferably 1-5 wt. %, based in each case on the overall composition.

Other preferred antiperspirant compositions according to the invention contain at least one active antiperspirant ingredient, at least 5 wt. % free water, 0.5-15 wt. % 1,4-dimethylolcyclohexane and in total 0.00001 to 10 wt. %, preferably 0.5-7 wt. %, particularly preferably 1-5 wt. %, of at least one more volatile odorant, which is preferably selected from alkyl isothiocyanates (alkyl mustard oils), butanedione, limonene, linalool, linalyl acetate, linalyl propionate, menthol, menthone, methyl-n-heptenone, phellandrene, phenylacetaldehyde, terpinyl acetate, citral, citronellal and mixtures of these odorants, all quantitative data being based on the total weight of the antiperspirant composition.

In another preferred embodiment, the compositions according to the invention contain at least one hair growth-inhibiting substance. Preferred compositions according to the invention contain at least one hair growth-inhibiting substance in a total quantity of 0.0001-5 wt. %, preferably 0.001-2 wt. %, particularly preferably 0.01-1 wt. %, and extraordinarily preferably 0.1-0.5 wt. %, based in each case on the active substance weight of the hair growth-inhibiting active ingredient/s and the total weight of the composition according to the invention.

### EXEMPLARY EMBODIMENTS

The following formulation examples are intended to explain the subject matter of the invention without limiting it thereto.

Antiperspirant sticks according to the invention based on an oil-in-water emulsion (quantitative data in wt. %)

| Example no. | 1.1 | 1.2 | 1.3 | 1.4 | 1.5 | 1.6 | 1.7 |
|---|---|---|---|---|---|---|---|
| Cutina ^{®} AGS | 2.5 | 2.5 | — | — | — | 2.5 | 2.5 |
| Cutina ^{®} EGMS | — | — | 2.5 | 2 | — | — | — |
| Cutina ^{®} PES | — | — | — | — | 2 | — | — |
| Cutina ^{®} FS45 | 3.5 | 3.5 | 3.5 | 3.5 | 3.5 | 3.5 | 3.5 |
| Eumulgin ^{®} B2 | 0.8 | 0.8 | 0.8 | 0.8 | 0.8 | 0.8 | 0.8 |
| Eumulgin ^{®} B3 | 0.8 | 0.8 | 0.8 | 0.8 | 0.8 | 0.8 | 0.8 |
| Diisopropyl | 6 | 6 | 6 | 6 | 6 | 6 | 6 |
| adipate | | | | | | | |
| Novata ^{®} AB | 4 | 4 | 4 | 4 | 4 | 4 | 4 |
| Cutina ^{®} CP | 5 | 5 | 5 | 5 | 5 | 5 | 5 |
| Cutina ^{®} HR | 4 | 4 | 4 | 4 | 4 | 4 | 4 |
| Kester Wax K62 | 5 | 5 | 5 | 5 | 5 | 5 | 5 |
| Locron ^{®} L (50% | 40 | 40 | 40 | 40 | 40 | 40 | 40 |
| ACH solution) | | | | | | | |
| Talcum | 10 | 10 | 10 | 10 | 10 | 10 | 10 |
| Perfume | 1.2 | 1.2 | 1.2 | 1.2 | 1.2 | 1.2 | 1.2 |
| Sensiva SC 50 | — | — | 0.6 | 0.6 | 0.6 | 0.6 | 0.6 |
| 1,4-Dimethylol- | 5 | 5 | 3 | 10 | 5 | 2 | 5 |
| cyclohexane | | | | | | | |
| 1,2-Propanediol | 5 | 5 | 7 | — | 5 | — | — |
| 1,3-Butylene | — | — | — | — | — | 8 | 5 |
| glycol | | | | | | | |
| Water, deionized | to 100 | to 100 | to 100 | to 100 | to 100 | to 100 | to 100 |

### Example 2

### Antiperspirant Emulsions (Oil-in-Water Emulsions) According to the Invention (Quantitative Data in wt. %)

| | 2.1 | 2.2 | 2.3 | 2.4 |
|---|---|---|---|---|
| ALUMINUM ZIRCONIUM | 23.7 | 23.7 | — | — |
| TETRACHLOROHYDREX GLY | | | | |
| ALUMINUM | — | — | 20.0 | 20.0 |
| CHLOROHYDRATE | | | | |
| STEARETH-2 | 2.4 | 2.4 | 2.3 | 2.3 |
| STEARETH-21 | 1.6 | 1.6 | 1.5 | 1.5 |
| PARFUM | 1.2 | 1.2 | 1.0 | 1.5 |
| PPG-15 STEARYL ETHER | 0.5 | 0.5 | 0.5 | 0.5 |
| ALUMINUM STARCH | 0.1 | 0.1 | 0.1 | 0.1 |
| OCTENYLSUCCINATE | | | | |
| TOCOPHERYL ACETATE | 0.5 | 0.5 | 0.5 | 0.5 |
| ISOPROPYL MYRISTATE | — | — | 0.3 | 0.3 |
| 1,4-Dimethylolcyclohexane | 5.0 | 2.0 | 5.0 | 2.0 |
| Water, deionized | to 100.0 | to 100.0 | to 100.0 | to 100.0 |

The emulsions 2.1-2.4 are packed into a roll-on-applicator.

Sprayable, translucent antiperspirant microemulsions (data in wt. %)

| | 3.1 | 3.2 | 3.3 | 3.4 | 3.5 | 3.6 | 3.7 | 3.8 | 3.9 |
|---|---|---|---|---|---|---|---|---|---|
| Plantaren ^{®} | 1.71 | 1.71 | — | 1.71 | 1.71 | — | 1.71 | 1.71 | 1.71 |
| 1200 | | | | | | | | | |
| Plantaren ^{®} | 1.14 | 1.39 | 2.40 | 1.14 | 1.39 | 2.40 | 1.14 | 1.39 | 1.39 |
| 2000 | | | | | | | | | |
| Glycerol | 0.71 | 0.71 | — | 0.71 | 0.71 | — | 0.71 | 0.71 | 0.71 |
| monooleate | | | | | | | | | |
| Dioctyl ether | 4.00 | 4.00 | 0.09 | 4.00 | 4.00 | 0.09 | 4.00 | 4.00 | 4.00 |
| Octyldodecanol | 1.00 | 1.00 | 0.02 | 1.00 | 1.00 | 0.02 | 1.00 | 1.00 | 1.00 |
| Perfume oil | 1.00 | 1.00 | 1.00 | 1.00 | 1.00 | 1.00 | 1.00 | 1.00 | 1.00 |
| Aluminum | 20.00 | 20.00 | 15.00 | — | — | — | 18.00 | 15.00 | 15.00 |
| chlorohydrate | | | | | | | | | |
| Aluminum | — | — | — | 20.0 | 20.0 | 20.0 | — | — | — |
| zirconium | | | | | | | | | |
| tetrachloro- | | | | | | | | | |
| hydrex gly | | | | | | | | | |
| 1,4- | 3.0 | 3.0 | 3.0 | 3.0 | 5.0 | 4.0 | 1.0 | 5.0 | 5.0 |
| Dimethylol- | | | | | | | | | |
| cyclohexane | | | | | | | | | |
| 1,2-Propylene | 2.0 | 2.0 | — | 2.0 | — | — | 4.0 | — | — |
| glycol | | | | | | | | | |
| Glycerol | — | — | 2.0 | — | — | 1.00 | — | — | — |
| Phenoxyethanol | 1.0 | — | — | — | 1.0 | 1.0 | — | — | — |
| Zinc lactate | — | 0.2 | — | — | — | — | — | — | — |
| Hinokitiol | — | — | 0.01 | — | — | — | — | — | — |
| Water | to | to | to | to | to | to | to | to | to |
| | 100 | 100 | 100 | 100 | 100 | 100 | 100 | 100 | 100 |

Sprayable antiperspirant microemulsions (data in wt. %)

| | 3.10 | 3.11 | 3.12 | 3.13 |
|---|---|---|---|---|
| Aluminum chlorohydrate | 30.00 | 40.00 | 35.00 | 40.00 |
| 50% solution | | | | |
| Dicaprylyl ether | 10.00 | 10.00 | 8.00 | 9.00 |
| 1,4-Dimethylolcyclohexane | 5.00 | 3.00 | 5.00 | 3.00 |
| Beheneth-10 | 3.30 | 4.00 | 3.50 | 4.00 |
| Cetearyl isononanoate | — | — | 4.00 | 5.00 |
| Hexyldecanol/hexyldecyl laurate | 3.00 | 5.00 | — | — |
| Parfum | 1.00 | 0.80 | 1.20 | 1.00 |
| Chamomile extract | 0.20 | — | 0.50 | — |
| Polysorbate 20/linoleic acid | 0.20 | 0.20 | 0.50 | — |
| Allantoin | 0.10 | — | — | 0.20 |
| Water | to 100 | to 100 | to 100 | to 100 |

The above-mentioned sprayable antiperspirant microemulsions 3.1 to 3.13 were applied onto the axillary skin both with a propellant-free pump spray and with a propellant. In addition, the antiperspirant microemulsions 3.1 and 3.13 were applied onto a nonwoven cloth and a cellulose wipe to obtain antiperspirant wipes.

Antiperspirant Roll-on (Data in Wt. %)

| | 4.1 | 4.2 | 4.3 |
|---|---|---|---|
| Ethanol, 96% (DEP denatured) | 25.0 | 20.0 | 30.0 |
| 1,4-Dimethylolcyclohexane | 5.0 | 10.0 | 5.0 |
| Mergital ® CS 11 | 2.0 | 2.0 | 2.0 |
| Eumulgin ® B 3 | 2.0 | 2.0 | 2.0 |
| Aluminum chlorohydrate | 20.0 | 20.0 | 20.0 |
| Hydroxyethyl cellulose | 0.5 | 0.5 | 0.5 |
| Dermosoft ® HMA | 0.5 | — | — |
| Perfume oil | 0.8 | 0.8 | 0.8 |
| Water | to 100 | to 100 | to 100 |

### Antiperspirant Wipes

### Examples Nos. 5.1-5.3

For the embodiment as an antiperspirant wipe according to the invention, in each case 75 g per square meter of example formulations 4.1 and 4.2 and 4.3 respectively were applied onto a single-layer substrate comprising 100% viscose with a basis weight of 50 g/m², which was cut into wipes of suitable size and packed in sachets.

Water-in-Oil Emulsion Sprays

| | Example | | | |
|---|---|---|---|---|
| | 6.1 | | | |
| | [wt. % | | Example 6.2 | |
| | based on | | [wt. % | |
| | total | Example 6.1 | based on | Example 6.2 |
| | prepara- | [wt. % | total | [wt. % |
| | tion | based on | preparation | based on |
| | incl. | W/O | incl. | W/O |
| | propellant] | emulsion] | propellant] | emulsion] |
| Dow Corning | 2.0 | 12.5 | 1.0 | 4.9 |
| 345 Fluid | | | | |
| 1,4- | 0.5 | 3.1 | 2.0 | 9.8 |
| Dimethylol- | | | | |
| cyclohexane | | | | |
| Dow Corning | 3.0 | 16.9 (oil) | 2.5 | 11.0 (oil) |
| 5225 C | | 1.9 | | 1.2 |
| | | (emulsifier) | | (emulsifier) |
| 2-Ethylhexyl | 0.5 | 3.1 | 0.5 | 2.4 |
| palmitate | | | | |
| Phenoxyethanol | 0.08 | 0.5 | 0.1 | 0.5 |
| Water, | 4.92 | 30.7 | 7.15 | 34.9 |
| deionized | | | | |
| Microdry | 5.0 | 31.3 | 7.25 | 35.3 |
| n-Butane | 84.0 | — | 79.5 | — |

W/O Emulsion Compact Spray

| | Example | | | |
|---|---|---|---|---|
| | 7.1 [wt. % | | Example | |
| | based on | | 7.2 [wt. % | |
| | total | Example 7.1 | based on | Example 7.2 |
| | prepara- | [wt. % | total | [wt. % |
| | tion | based on | preparation | based on |
| | incl. | W/O | incl. | W/O |
| | propellant] | emulsion] | propellant] | emulsion] |
| Dow Corning | 0.48 | 1.6 | 2.173 | 4.1 |
| 345 Fluid | | | | |
| 1,4-Dimethylol- | 1.5 | 5.0 | 4.823 | 9.1 |
| cyclohexane | | | | |
| Dow Corning | 4.2 | 12.53 | 7.42 | 12.53 |
| 5225 C | | (cyclo- | | (cyclo- |
| | | methicone) | | methicone) |
| | | 1.47 | | 1.47 |
| | | (emulsifier) | | (emulsifier) |
| Benzoic acid | 1.38 | 4.6 | — | — |
| C12-15 alkyl | | | | |
| ester | | | | |
| 2-Ethylhexyl | — | — | 1.192 | 2.25 |
| palmitate | | | | |
| Parfum | 0.75 | 2.5 | — | — |
| Phenoxyethanol | 0.15 | 0.5 | 0.265 | 0.5 |
| Water, deionized | 1.74 | 5.8 | 2.15 | 4.05 |
| Aluminum | 19.8 | 66.0 | 34.98 | 66.0 |
| chlorohydrate, | | | | |
| 50% aqueous | | | | |
| solution | | | | |
| n-Butane | 70.0 | — | 47.0 | — |

Exemplary composition 7.1 contains an antiperspirant emulsion according to the invention with 9.9 wt. % aluminum chlorohydrate, based on the weight of the propellant-free emulsion. At a spraying rate of 0.35 g/s, 0.035 g of the active antiperspirant ingredient aluminum chlorohydrate are sprayed onto the skin surface per second.

Exemplary composition 7.2 contains an antiperspirant emulsion according to the invention with 17.49 wt. % aluminum chlorohydrate, based on the weight of the propellant-free emulsion. At a spraying rate of 0.2 g/s, 0.035 g of the active antiperspirant ingredient aluminum chlorohydrate are sprayed onto the skin surface per second.

Water-in-Oil Emulsion for Administration as a Spray (with Propellant) (Data in Wt. %)

| | Example 8.1 |
|---|---|
| Aluminum chlorohydrate | 33 |
| C₁₀-C₁₃ isoalkane | 8.9 |
| PEG/PPG-18/18 Dimethicone | 1.4 |
| Isoceteth-20 | 0.5 |
| Dimethicone (5 cSt) | 4.2 |
| Isopropyl myristate | 9.0 |
| 1,2-Propanediol | 5.0 |
| 1,4-Dimethylolcyclohexane | 4.0 |
| Phenoxyethanol | 0.50 |
| Perfume | 2.5 |
| Water | to 100 |

Clear Antiperspirant Liquids and Antiperspirant Gels (Data in Wt. %)

| | 9.1 | 9.2 | 9.3 | 9.4 |
|---|---|---|---|---|
| 1,2-Propylene glycol | 12.80 | 15.8 | 12.8 | 9.6 |
| 1,4- | 5.0 | 7.0 | 5.0 | 10.0 |
| Dimethylolcyclohexane | | | | |
| Aluminum | 40.00 | 40.00 | 40.00 | 40.00 |
| chlorohydrate, 50% | | | | |
| solution in water | | | | |
| Cyclopentasiloxane | 14.20 | 14.20 | 14.20 | 14.20 |
| Ethanol denat. | 5.00 | 10.00 | 8.00 | 10.00 |
| Abil EM 97 | 3.50 | 2.50 | 3.20 | 3.00 |
| Parfum | 0.60 | 0.60 | 1.00 | 1.30 |
| Panthenol | 0.50 | — | — | — |
| Water | to 100 | to 100 | to 100 | to 100 |

To achieve clear compositions, the refractive index of the aqueous phase was matched to the refractive index of the oil phase to an accuracy of ±0.0002. Water or propylene glycol was used as a variable.

When mixed together at a moderate stirring rate (400 revolutions per minute), clear antiperspirant liquids were initially formed. Subsequent stirring at 900 revolutions per minute and shearing with a homogenizer (Ultra Turrax T50 from IKA-Werke, stator diameter 4 cm, rotor diameter 3.2 cm, single rotor, single opening, gap width 4 mm, stirring rate: step 8 (approx. 8500 rpm) for 90 seconds gave gels with a viscosity of about 120000 mPas (Brookfield RVF with Helipath, spindle: TC, 4 revolutions per minute, temperature: 20° C.).

Antiperspirant Cream Based on an Oil-in-Water Emulsion (Data in Wt. %)

| | 10.1 | 10.2 | 10.3 | 10.4 |
|---|---|---|---|---|
| Aluminum chlorohydrate 50% | 40.00 | 40.00 | 35.00 | 45.00 |
| solution in water | | | | |
| 1,4-Dimethylolcyclohexane | 7.0 | 2.0 | 5.0 | 10.0 |
| Glyceryl stearate | 5.00 | 4.50 | 5.50 | 6.00 |
| Cetyl alcohol | 2.00 | — | 3.00 | 1.50 |
| Behenyl alcohol | 1.50 | 4.00 | 3.50 | 5.00 |
| Dimethicone (350 cSt) | 2.00 | 1.50 | 2.50 | 3.00 |
| Ceteareth-12 | 1.50 | 2.00 | 2.50 | 1.30 |
| Ceteareth-20 | 1.50 | 2.00 | 2.50 | 1.30 |
| Cetiol ® PGL | 3.00 | 4.00 | 2.50 | 2.40 |
| Cyclopentasiloxane | 1.50 | 3.00 | 2.00 | 1.00 |
| Tocopheryl acetate | 0.05 | 0.25 | — | — |
| Perfume | 0.80 | 1.00 | 1.50 | 2.00 |
| Allantoin | 0.10 | 0.10 | — | — |
| Phenoxyethanol | 0.50 | 0.50 | 0.50 | 0.50 |
| Water | to 100 | to 100 | to 100 | to 100 |

Translucent Antiperspirant Stick Based on a Structured Microemulsion According to WO 02/083091 A1 (Data in Wt. %)

| | Example 11.1 |
|---|---|
| Cyclomethicone | 27.9 |
| Isoeicosane | 5.0 |
| Lanette O | 8.4 |
| Cetyl octanoate | 6.2 |
| Brij 78 | 9.5 |
| Microdry | 19.0 |
| 1,4-Dimethylolcyclohexane | 4.0 |
| N-Lauroyl-L-glutamic acid di-n- | 5.0 |
| butylamide (GP-1 from Ajinomoto, m.p. | |
| 155-163° C.) | |
| Water | 15.0 |

Water-in-Oil Emulsion Antiperspirant Stick

| | Example 12.1 |
|---|---|
| Dow Corning 345 Fluid | 23.7 |
| Finsolv TN | 12.6 |
| Isostearyl alcohol | 11.5 |
| N-Lauroyl-L-glutamic acid di-n- | 3.8 |
| butylamide (GP-1 from Ajinomoto, m.p. | |
| 155-163° C.) | |
| Abil EM 90 | 0.95 |
| Aluminum zirconium tetrachlorohydrex | 20 |
| gly | |
| Glycerol, 99.5 wt. % | 5.0 |
| 1,4-Dimethylolcyclohexane | 5.0 |
| Water | 20.0 |

The exemplary compositions according to the invention are applied onto the skin, in particular the axillary skin.

The present application thus also provides a method of non-therapeutic perspiration reduction, wherein a composition according to the invention or which is preferred according to the invention is applied onto the skin, in particular the axillary skin.

With regard to further preferred embodiments of the method according to the invention, the statements made above regarding the compositions according to the invention apply mutatis mutandis.

List of Raw Materials Used

| | | Supplier/ |
|---|---|---|
| Component | INCI | manufacturer |
| Abil EM 90 | Cetyl PEG/PPG-10/1 Dimethicone | Evonik |
| | | Degussa |
| Abil EM 97 | Bis-PEG/PPG-14/14 Dimethicone | Evonik |
| | | Degussa |
| Brij 78 | Steareth-20 | Uniqema |
| Cetiol ® OE | DICAPRYLYL ETHER | Cognis |
| Cetiol ® PGL | Hexyldecanol/Hexyldecyl Laurate | Cognis |
| Cutina ® AGS | Glycol Distearate | Cognis |
| Cutina ® EGMS | Glycol Stearate | Cognis |
| Cutina ® PES | Pentaerythrityl Distearate | Cognis |
| Cutina ® CP | Cetyl Palmitate | Cognis |
| Cutina ® FS45 | Palmitic Acid, Stearic Acid | Cognis |
| Cutina ® HR | Hydrogenated Castor Oil | Cognis |
| Cutina ® E 24 PF | PEG-20 GLYCERYL STEARATE | Cognis |
| DC ® 245 | Cyclopentasiloxane | Dow |
| | | Corning |
| Dermosoft HMA | p-Hydroxymandelic acid sodium | Dr. |
| | salt, active substance 95-96% | Straetmans |
| Dow Corning 345 | Cyclomethicone | Dow |
| Fluid | (decamethylcyclopentasiloxane, | Corning |
| | dodecamethylcyclohexasiloxane) | |
| Dow Corning 5225 | Cyclomethicone, PEG/PPG-18/18 | Dow |
| C Formulation Aid | Dimethicone in a weight ratio of 9:1 | Corning |
| Dow Corning ES | Dimethicone (5 cSt), PEG/PPG- | Dow |
| 5227 DM | 18/18 Dimethicone in a weight ratio | Corning |
| Formulation Aid | of 3:1 | |
| Eumulgin ® B2 | Ceteareth-20 | Cognis |
| Eumulgin ® B3 | Ceteareth-30 | Cognis |
| Eumulgin ® B 3 | CETEARETH-30 | Cognis |
| Eutanol ® G | Octyldodecanol | Cognis |
| Eutanol ® G 16 | 2-Hexyldecanol | Cognis |
| Finsolv TN | C12-15 ALKYL BENZOATE | Innospec |
| Kester Wax K62 | Cetearyl Behenate | Koster |
| | | Keunen |
| Lanette ® O | Cetyl/Stearyl alcohol in a ratio of | Cognis |
| | 1:1 | |
| Locron L (50% | Aluminum chlorohydrate | Clariant |
| ACH solution) | | |
| Lorol ® C 18 | Stearyl alcohol | Cognis |
| Mergital ® CS 11 | CETEARETH-11 | Cognis |
| Microdry ® | Aluminum chlorohydrate | Reheis |
| Novata ® AB | Cocoglycerides | Cognis |
| Plantaren ® 1200 | LAURYL GLUCOSIDE, approx. | Cognis |
| | 50% active substance | |
| Plantaren ® 2000 | DECYL GLUCOSIDE, approx. | Cognis |
| | 50% active substance | |
| Sensiva ® SC 50 | 2-Ethylhexyl glycerol ether | Schülke |
| | | & Mayr |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An antiperspirant composition, comprising:
a) at least one active antiperspirant ingredient,
b) at least 5 wt. % free water, and
c) 0.5-15 wt. % 1,4-dimethylolcyclohexane,
wherein all quantitative data correspond to the total weight of the antiperspirant composition, without taking into account any added propellant.

2. The composition according to claim 1 wherein the 1,4-dimethylolcyclohexane is present as a cis/trans isomer mixture.

3. The composition according to claim 1 wherein the antiperspirant composition, based on the total weight thereof, includes 10 to 95 wt. % free water.

4. The composition according to claim 1 wherein the antiperspirant composition, based on the total weight thereof, includes 1 to 12 wt 1,4-dimethylolcyclohexane.

5. The composition according to claim 1 wherein the active antiperspirant ingredient is selected from the group consisting of water-soluble astringent inorganic and organic salts of aluminum, zirconium and zinc or any mixtures of these salts, and is included in a quantity of 5-40 wt. % based on the total weight of the active substance (USP) free from water of crystallization in the overall composition.

6. The composition according to claim 1 wherein at least one odorant component is included in a total quantity of 0.00001 to 10 wt. % based in each case on the overall composition.

7. The composition according to claim 1 wherein 0.00001 to 10 wt. % of at least one more volatile odorant is included, which is selected from the group consisting of alkyl isothiocyanates (alkyl mustard oils), butanedione, limonene, linalool, linalyl acetate, linalyl propionate, menthol, menthone, methyl-n-heptenone, phellandrene, phenyl acetaldehyde, terpinyl acetate, citral, citronellal and mixtures of such odorants, all quantitative data being based on the total weight of the antiperspirant composition.

8. The composition according to claim 1 wherein the composition is present in stick form, as an aerosol spray, pump spray, liquid or gel roll-on application, cream, lotion, solution, gel or applied on a substrate.

9. The composition according to claim 1, wherein the composition is present as an oil-in-water emulsion and comprises at least one nonionic emulsifier with an HLB value in the range of 3-6 in a total quantity of 1.8-3 wt. % and further comprises at least one nonionic emulsifier with an HLB value in the range of 12-18 in a total quantity of 1-2 wt. %, the quantitative data being based in each case on the total weight of the composition.

10. The composition according to claim 1, wherein the composition is present as a water-in-oil emulsion and comprises at least one water-in-oil emulsifier, and at least one nonionic polyalkylene glycol ether with an HLB value >7 and selected from the group consisting of isoceteth-20, and at least one C₈-C₁₆ isoparaffin.

11. The composition according to claim 1, further comprising at least one hydrogel former selected from the group consisting of hydroxyalkyl celluloses, xanthan gum, sclerotium gum, succinoglucans, polygalactomannans pectins, agar, carragheen (carrageenan), tragacanth, gum arabic, karaya gum, tara gum, gellan, gelatin, casein, pectin, propylene glycol alginate, algic acids and salts thereof, polyvinylpyrrolidones, polyvinyl alcohols, polyacrylamides, and also physically and/or chemically modified starches, water-soluble acrylic acid-acrylate copolymers, acrylic acid-acrylamide copolymers, acrylic acid-vinylpyrrolidone copolymers, acrylic acid-vinyl formamide copolymers and polyacrylates.

12. The composition according to claim 1, wherein the formulation is formulated as an oil-in-water emulsion, which does not represent a microemulsion, and comprises
0.5-15 wt. % 1,4-dimethylolcyclohexane,
0.5-6.5 wt. % oil or fat phase, encompassing at least one oil component which is liquid at 20° C., selected from
linear and branched saturated mono- or polyhydric C₃-C₃₀ alkanols, which are etherified with at least one propylene oxide unit per molecule,
propylene glycol monoesters of branched saturated C₆-C₃₀ alkane carboxylic acids and
branched saturated C₁₀-C₃₀ alkanols,
at least 60 wt. % being the free water,
the at least one active antiperspirant ingredient, and
at least one polysaccharide,
wherein the composition is suitable for application with a roller ball applicator, the quantitative data being based in each case on the total weight of the composition according to the invention, without taking into account any added propellant.

13. The composition according to claim 1, wherein the composition is present in the form of a water-in-oil emulsion and, based in each case on its propellant-free total weight, comprises the at least one active antiperspirant ingredient, 10-85 wt. % the free water, 0.5-15 wt. % the 1,4-dimethylolcyclohexane, at least one water-in-oil emulsifier, and 1-40 wt. % at least one cosmetic oil, selected from two- and three-component mixtures of C₈-C₁₆ isoalkanes, linear dimethyl polysiloxanes with kinematic viscosities in the range of 5-100 cSt (25° C.) and esters of linear or branched saturated or unsaturated fatty alcohols with 2-30 carbon atoms with linear or branched saturated or unsaturated fatty acids with 2-30 carbon atoms, which may be hydroxylated, and is packed with at least one propellant in an aerosol dispensing device.

14. The composition according to claim 13, wherein at least one nonionic polyalkylene glycol ether with an HLB value >7 is included in a total quantity of 0.1-3 wt. % based in each case on the total weight of the propellant-free antiperspirant composition.

15. The composition according to claim 1, wherein the composition is present as a water-in-oil emulsion in gel form, comprises the at least one active antiperspirant ingredient, at least 5 wt. % the free water, 0.5-15 wt. % the 1,4-dimethylolcyclohexane, at least one water-in-oil emulsifier, and 1-40 wt. % at least one cosmetic oil, the quantitative data being based in each case on the total weight of the antiperspirant composition, and has a dynamic viscosity at 25° C. in the range of 15,000 to 300,000 mPas.
